(19) United States
(12) Patent Application Publication
(10) Pub. No.: US 2016/0237106 A1
(43) Pub. Date: Aug. 18, 2016

(54) 4'-AZIDO SUBSTITUTED NUCLEOSIDE DERIVATIVES AS INHIBITORS OF EBOLA VIRUS RNA REPLICATION

(71) Applicant: Riboscience LLC, Palo Alto, CA (US)

(72) Inventors: Mark Smith, San Francisco, CA (US); Klaus G. Klumpp, West Chester, PA (US)

(21) Appl. No.: 14/994,520

(22) Filed: Jan. 13, 2016

Related U.S. Application Data
(60) Provisional application No. 62/103,330, filed on Jan. 14, 2015.

(51) Int. Cl. C07H 19/06; C07H 19/23

(52) U.S. Cl. CPC C07H 19/06; C07H 19/23

(57) ABSTRACT

The application discloses compounds of Formula I

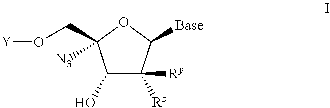

wherein the variable substituents are as defined herein. The compounds of Formula I and pharmaceutical compositions comprising compounds of Formula I are useful for the treatment of diseases mediated by Filoviruses such as Ebolavirus.

### CROSS-REFERENCE TO RELATED APPLICATIONS

This application claims priority to U.S. provisional application Ser. No. 62/103,330, filed Jan. 14, 2015, the disclosure of which is incorporated herein by reference and is commonly owned.

### FIELD OF THE INVENTION

The invention relates to nucleoside derivatives as inhibitors of Ebola Virus replication. In particular, the invention is concerned with the use of purine and pyrimidine nucleoside derivatives as inhibitors of replication of Ebola and related negative strand and positive strand RNA viruses and pharmaceutical compositions containing such compounds.

Ebola virus is a member of the Filoviridae family of viruses (members are called Filoviruses), which includes Ebolavirus and Marburgvirus genera. Infection of humans with Ebola or Marburg viruses commonly results in the development of severe hemorrhagic fever with a high mortality rate. Initial symptoms include fever, sore throat, headaches and general muscle pain. This is followed by vomiting and diarrhea with internal and external bleeding. Human to human transmission is effective through contact with body fluids.

Many of the drugs approved for the treatment of viral infections are nucleosides or nucleoside analogues and most of these nucleoside analogue drugs inhibit viral replication, following conversion to the corresponding triphosphates, through inhibition of the viral polymerase enzymes. Inhibition of Filovirus polymerase is expected to prevent the production of infectious viral particles. Currently, there are very few examples of nucleosides or nucleoside monophosphate prodrugs that have been shown to effectively inhibit the production of Filoviruses.

Filoviruses are negative strand RNA viruses. Viruses with RNA dependent RNA polymerases closely related to Filovirus polymerase include negative strand RNA viruses of the Bornaviridae, Rhabdoviridae, Paramyxoviridae, Arenaviridae, Bunyaviridae and Orthomyxoviridae. Viruses with RNA dependent RNA polymerases also related to Filovirus polymerases include positive strand RNA viruses of the Coronaviridae, Picomaviridae, Flaviviridae and Togaviridae families.

### SUMMARY OF THE INVENTION

The compounds of Formula I are useful for the treatment of diseases mediated by Ebola and Marburg virus and related RNA viruses and for pharmaceutical compositions comprising such compounds.

The application provides a method of treating a disease mediated by Ebolavirus, Marburgvirus, or other Filovirus, comprising administering to a patient in need thereof a compound of Formula I

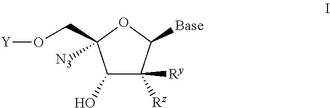

wherein:

### Y is H or P(═X)(R′)(R);

R is O—R¹ or NHR^{1′};
R^{1′} is —C(R^{2a})(R^{2b})C(═O)OR³;

R′ is N(R⁴)C(R^{2a})(R^{2b})C(═O)OR³, —OP(═O)(OH)OH, —OP(═O)(OH)OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
   each R^{1a} is independently H or lower alkyl;
   each R^{1b} is independently —OR^{1a} or —N(R^{1a})₂;
   each R^{1c} is lower alkyl;
   each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
      m is 0, 1, or 2;
      n is 1, 2, or 3;
      p is 1 or 2;
      r is 1 or 2;
   or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂;
each R⁴ is independently H, lower alkyl;
or R^{2b} and R⁴ together form (CH₂)₃;
   R^{y}, and R^{z} are each independently H, Me, OH or F; and
   Base is uracil, cytosine, guanine, adenine, thymine, or heterocycloalkyl, each of which may optionally be substituted with one or more hydroxy, lower alkyl, lower alkoxy, halo, nitro or cyano;
   or a pharmacologically acceptable salt thereof.

The compounds of Formula I, and pharmaceutical compositions thereof, are useful for the treatment of diseases mediated by Filoviruses such as Ebolavirus and Marburgvirus.

The application provides a method for treating an Ebolavirus or Marburgvirus, or other Filovirus, infection comprising administering to a patient in need thereof a therapeutically effective amount of a compound of Formula I.

### DETAILED DESCRIPTION OF THE INVENTION

The compounds of Formula I have been shown to be inhibitors of Ebola Virus replication in an antiviral assay system based on the infection of Vero-E6 cells with infectious Ebola virus. These compounds target the viral RNA dependent RNA polymerase active site and have the potential to be efficacious as antiviral drugs for the treatment of Filovirus infections in human.

### DEFINITIONS

The term “alkyl” as used herein denotes a straight or branched chain hydrocarbon residue containing 1 to 12 carbon atoms. Preferably, the term “alkyl” denotes a straight or branched chain hydrocarbon residue containing 1 to 7 carbon atoms. Most preferred are methyl, ethyl, propyl, isopropyl, n-butyl, isobutyl, tert.-butyl or pentyl. The alkyl may be unsubstituted or substituted. The substituents are selected from one or more of cycloalkyl, nitro, amino, alkyl amino, dialkyl amino, alkyl carbonyl and cycloalkyl carbonyl.

The term “cycloalkyl” as used herein denotes an optionally substituted cycloalkyl group containing 3 to 7 carbon atoms, e. g. cyclopropyl, cyclobutyl, cyclopentyl, cyclohexyl or cycloheptyl.

The term “alkoxy” as used herein denotes an optionally substituted straight or branched chain alkyl-oxy group wherein the “alkyl” portion is as defined above such as methoxy, ethoxy, n-propyloxy, i-propyloxy, n-butyloxy, i-butyloxy, tert.-butyloxy, pentyloxy, hexyloxy, heptyloxy including their isomers.

The term “alkoxyalkyl” as used herein denotes an alkoxy group as defined above which is bonded to an alkyl group as defined above. Examples are methoxymethyl, methoxyethyl, methoxypropyl, ethoxymethyl, ethoxyethyl, ethoxypropyl, propyloxypropyl, methoxybutyl, ethoxybutyl, propyloxybutyl, butyloxybutyl, tert.-butyloxybutyl, methoxypentyl, ethoxypentyl, propyloxypentyl including their isomers.

The term “alkenyl” as used herein denotes an unsubstituted or substituted hydrocarbon chain radical having from 2 to 7 carbon atoms, preferably from 2 to 4 carbon atoms, and having one or two olefinic double bonds, preferably one olefinic double bond. Examples are vinyl, 1-propenyl, 2-propenyl (allyl) or 2-butenyl (crotyl).

The term “alkynyl” as used herein denotes to unsubstituted or substituted hydrocarbon chain radical having from 2 to 7 carbon atoms, preferably 2 to 4 carbon atoms, and having one or where possible two triple bonds, preferably one triple bond. Examples are ethynyl, 1-propynyl, 2-propynyl, 1-butynyl, 2-butynyl or 3-butynyl.

The term “hydroxyalkyl” as used herein denotes a straight or branched chain alkyl group as defined above wherein 1, 2, 3 or more hydrogen atoms are substituted by a hydroxy group. Examples are hydroxymethyl, 1-hydroxyethyl, 2-hydroxyethyl, 1-hydroxypropyl, 2-hydroxypropyl, 3-hydroxypropyl, hydroxyisopropyl, hydroxybutyl and the like.

The term “haloalkyl” as used herein denotes a straight or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl and the like.

The term “alkylthio” as used herein denotes a straight or branched chain (alkyl)S— group wherein the “alkyl” portion is as defined above. Examples are methylthio, ethylthio, n-propylthio, i-propylthio, n-butylthio, i-butylthio or tert.-butylthio.

The term “aryl” as used herein denotes an optionally substituted phenyl and naphthyl (e. g. 1-naphthyl, 2-naphthyl or 3-naphthyl). Suitable substituents for aryl can be selected from those named for alkyl, in addition however, halogen, hydroxy and optionally substituted alkyl, haloalkyl, alkenyl, alkynyl and aryloxy are substituents which can be added to the selection.

The term “heterocyclyl” or “heterocycloalkyl” as used herein denotes an optionally substituted saturated, partially unsaturated or aromatic monocyclic, bicyclic or tricyclic heterocyclic systems which contain one or more hetero atoms selected from nitrogen, oxygen and sulfur which can also be fused to an optionally substituted saturated, partially unsaturated or aromatic monocyclic carbocycle or heterocycle.

Examples of suitable heterocycles are oxazolyl, isoxazolyl, furyl, tetrahydrofuryl, 1,3-dioxolanyl, dihydropyranyl, 2-thienyl, 3-thienyl, pyrazinyl, isothiazolyl, dihydrooxazolyl, pyrimidinyl, tetrazolyl, 1-pyrrolidinyl, 2-pyrrolidinyl, 3-pyrrolidinyl, pyrrolidinonyl, (N-oxide)-pyridinyl, 1-pyrrolyl, 2-pyrrolyl, triazolyl e. g. 1,2,3-triazolyl or 1,2,4-triazolyl, 1-pyrazolyl, 2-pyrazolyl, 4-pyrazolyl, piperidinyl, morpholinyl (e. g. 4-morpholinyl), thiomorpholinyl (e. g. 4-thiomorpholinyl), thiazolyl, pyridinyl, dihydrothiazolyl, imidazolidinyl, pyrazolinyl, piperazinyl, 1-imidazolyl, 2-imidazolyl, 4-imidazolyl, thiadiazolyl e. g. 1,2,3-thiadiazolyl, 4-methylpiperazinyl, 4-hydroxypiperidin-1-yl.

Suitable substituents for heterocycloalkyl can be selected from those named for alkyl, in addition however, optionally substituted alkyl, alkenyl, alkynyl, an oxo group (═O) or aminosulphonyl are substituents which can be added to the selection.

The term “acyl” (“alkylcarbonyl”) as used herein denotes a group of formula C(═O)R wherein R is hydrogen, an unsubstituted or substituted straight or branched chain hydrocarbon residue containing 1 to 7 carbon atoms or a phenyl group. Most preferred acyl groups are those wherein R is hydrogen, an unsubstituted straight chain or branched hydrocarbon residue containing 1 to 4 carbon atoms or a phenyl group.

The term halogen refers to fluorine, chlorine, bromine or iodine, but preferably fluorine, chlorine, bromine.

In the pictorial representation of the compounds given throughout this application, a thickened tapered line ( [symbol] ) indicates a substituent which is above the plane of the ring to which the asymmetric carbon belongs and a dotted line ( [symbol] ) indicates a substituent which is below the plane of the ring to which the asymmetric carbon belongs.

Compounds of formula I and II exhibit stereoisomerism. These compounds can be any isomer of the compound of formula I or mixtures of these isomers. The compounds and intermediates of the present invention having one or more asymmetric carbon atoms may be obtained as racemic mixtures of stereoisomers which can be resolved.

Compounds of formulae I and II may exhibit tautomerism meaning that the compounds of this invention can exist as two or more chemical compounds that are capable of facile interconversion. In many cases it merely means the exchange of a hydrogen atom between two other atoms, to either of which it forms a covalent bond. Tautomeric compounds exist in a mobile equilibrium with each other, so that attempts to prepare the separate substances usually result in the formation of a mixture that shows all the chemical and physical properties to be expected on the basis of the structures of the components.

The most common type of tautomerism is that involving carbonyl, or keto, compounds and unsaturated hydroxyl compounds, or enols. The structural change is the shift of a hydrogen atom between atoms of carbon and oxygen, with the rearrangement of bonds. For example, in many aliphatic aldehydes and ketones, such as acetaldehyde, the keto form is the predominant one; in phenols, the enol form is the major component.

Compounds of formulae I and II which are basic can form pharmaceutically acceptable salts with inorganic acids such as hydrohalic acids (e.g. hydrochloric acid and hydrobromic acid), sulphuric acid, nitric acid and phosphoric acid, and the like, and with organic acids (e.g. with acetic acid, tartaric acid, succinic acid, fumaric acid, maleic acid, malic acid, salicylic acid, citric acid, methanesulphonic acid and p-toluene sulphonic acid, and the like). The formation and isolation of such salts can be carried out according to methods known in the art.

### Inhibitors of Filoviruses

The application provides a method of treating a disease mediated by Ebolavirus, Marburgvirus, or other Filovirus, comprising administering to a patient in need thereof a therapeutically effective amount of a compound of Formula I

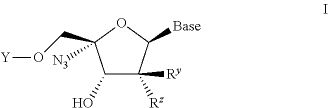

wherein:

### Y is H or P(═X)(R′)(R);

R is O—R¹ or NHR^{1′};
R^{1′} is —C(R^{2a})(R^{2b})C(═O)OR³;

R′ is N(R⁴)C(R^{2a})(R^{2b})C(═O)OR³, —OP(═O)(OH)OH, —OP(═O)(OH)OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
   each R^{1a} is independently H or lower alkyl;
   each R^{1b} is independently —OR^{1a} or —N(R^{1a})₂;
   each R^{1c} is lower alkyl;
   each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
      m is 0, 1, or 2;
      n is 1, 2, or 3;
      p is 1 or 2;
      r is 1 or 2;
   or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂;
each R⁴ is independently H, lower alkyl;
or R^{2b} and R⁴ together form (CH₂)₃;
   R^{y}, and R^{z} are each independently H, Me, OH or F; and
   Base is uracil, cytosine, guanine, adenine, thymine, or heterocycloalkyl, each of which may optionally be substituted with one or more hydroxy, lower alkyl, lower alkoxy, halo, nitro or cyano;
   or a pharmacologically acceptable salt thereof.

The application provides the above method, wherein the Filovirus is Marburgvirus

The application alternatively provides the above method, wherein the Filovirus is Ebolavirus.

The application provides any of the above methods, wherein R^{y} is H.

The application provides any of the above methods, wherein R^{z} is F.

The application provides any of the above methods, wherein Y is H.

The application provides any of the above methods, wherein Base is guanine.

The application provides a method of treating a disease mediated by Ebolavirus, comprising administering to a patient in need thereof a therapeutically effective amount of a compound of Formula I, wherein R^{y} is H and R^{z} is F.

The application provides the above method, wherein Y is H.

The application provides either of the above methods, wherein Base is guanine.

The application provides a compound of Formula II,

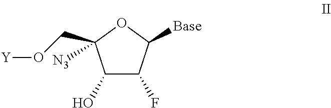

wherein:
Base is guanine;

### Y is P(═X)(R′)(R);

R is O—R¹ or NHR^{1′};
R^{1′} is —C(R^{2a})(R^{2b})C(═O)OR³;

R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
   each R^{1a} is independently H or lower alkyl;
   each R^{1b} is independently —OR^{1a} or —N(R^{1a})₂;
   each R^{1c} is lower alkyl;
   each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
      m is 0, 1, or 2;
      n is 1, 2, or 3;
      p is 1 or 2;
      r is 1 or 2;
   or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and
each R⁴ is independently H, lower alkyl;
or R^{2b} and R⁴ together form (CH₂)₃;
   with the proviso that the compound is not cyclohexyl ((((2R,3R,4R,5R)-5-(2-amino-6-oxo-3,6-dihydro-9H-purin-9-yl)-2-azido-4-fluoro-3-hydroxytetrahydrofuran-2-yl)methoxy)(phenoxy)phosphoryl)-L-alaninate;
   or a pharmacologically acceptable salt thereof.

The application provides the above compound of Formula II, wherein R⁴ is H.

The application provides either of the above compounds of Formula II, wherein R¹ is naphthyl or phenyl.

The application provides any one of the above compounds of Formula II, wherein R^{2a} is H.

The application provides any one of the above compounds of Formula II, wherein R^{2b} is methyl.

The application provides any one of the above compounds of Formula II, wherein R³ is isopropyl.

The application provides any one of the above compounds of Formula II, wherein R′ is —OR³ and R³ is isopropyl.

The application provides a composition comprising any one of the above compounds of Formula II.

The application provides the above composition, admixed with at least one carrier, diluent or excipient.

The application provides a method of treating a disease mediated by a Filovirus, comprising administering to a patient in need thereof a therapeutically effective amount of a compound of Formula I, wherein the compound of Formula I is selected from the group consisting of:
4′-Azido-2′-deoxy-2′-fluorouridine;
4′-Azidouridine
4′-Azido-2′-deoxy-2′-fluorouridine-5′-(O-phenyl-N-(S)-1-(isopropoxycarbonyl)ethyl phosphoramidate
4′-Azidouridine-5′-(O-phenyl-N-(S)-1-(isopropoxycarbonyl)ethyl phosphoramidate;
4′-Azido-2′-deoxy-2′-fluorouridine-5′-(O-1-naphthyl-N-(S)-1-(isopropoxycarbonyl)ethyl phosphoramidate;
4′-Azidouridine-5′-(O-1-naphthyl-N-(S)-1-(benzyloxycarbonyl)ethyl phosphoramidate
4′-Azido-2′-deoxy-2′-fluorouridine-5′-(O-1-naphthyl-N-(S)-1-(i2,2-dimethylpropoxycarbonyl)ethyl phosphoramidate;
4′-Azido-2′-deoxy-2′-fluorouridine-5′-(O-1-naphthyl-N-(S)-1-(benzyloxycarbonyl)ethyl phosphoramidate;
4′-Azido-2′-deoxy-2′-fluorouridine-5′-(O-1-phenyl-N-(S)-1-(hexoxycarbonyl)ethyl phosphoramidate;
4′-Azido-2′-deoxy-2′-fluorouridine-5′-(O-1-naphthyl-N-(S)-1-(cyclopentoxycarbonyl)ethyl phosphoramidate;
4′-Azido-2′-deoxy-2′-fluorouridine-5′-{N,N′-bis[(S)-1-(i sopropoxycarbonyl)ethyl]phosphorodiamidate};
4′-Azido-2′-deoxy-2′-fluorouridine-5′-{N,N′-bis[(S)-1-(benzyloxycarbonyl)ethyl]phosphorodiamidate};
4′-Azido-2′-deoxy-2′-fluorouridine-5′-{N,N′-bis[(S)-1-(hexoxycarbonyl)ethyl]phosphorodiamidate};
4′-Azido-2′-deoxy-2′-fluorouridine-5′-{N,N′-bis[(S)-1-(cyclopentoxycarbonyl)ethyl]phosphorodiamidate};
4′-Azido-2′-deoxy-2′-fluorouridine-5′-O-(2-oxido-4-H-1,3,2-benzodioxaphosphorin-2-yl)-uridine;
4′-Azido-2′-deoxy-2′-fluorouridine-5′-O-[bis(4-methoxyphenoxy)phosphinyl]uridine;
4′-Azido-2′-deoxy-2′-fluorocytidine;
4′-Azidocytidine;
4′-Azido-2′-deoxy-2′-fluorocytidine-5′-(O-phenyl-N-(S)-1-(ethoxycarbonyl)ethyl phosphoramidate;
4′-Azidocytidine-5′-(O-phenyl-N-(S)-1-(benzyloxycarbonyl)ethyl phosphoramidate;
4′-Azido-2′-deoxy-2′-fluorocytidine-5′-(O-phenyl-N-(S)-1-(isopropoxycarbonyl)ethyl phosphoramidate;
4′-Azidocytidine-5′-(O-phenyl-N-(S)-1-(eisopropoxycarbonyl)ethyl phosphoramidate;
4′-Azido-2′-deoxy-2′-fluorocytidine-5′-(O-phenyl-N-(S)-1-(neopentoxycarbonyl)ethyl phosphoramidate;
4′-Azido-2′-deoxy-2′-fluorocytidine-5′-(O-1-naphthyl-N-(S)-1-(isopropoxycarbonyl)ethyl phosphoramidate;
4′-Azido-2′-deoxy-2′-fluorocytidine-5′-(O-1-naphthyl-N-(S)-1-(2,2-dimethylpropoxycarbonyl)ethyl phosphoramidate;
4′-Azido-2′-deoxy-2′-fluorocytidine-5′-(O-1-naphthyl-N-(S)-1-(benzyloxycarbonyl)ethyl phosphoramidate;
4′-Azidocytidine-5′-(O-naphthyl-N-(S)-1-(benzyloxycarbonyl)ethyl phosphoramidate;
4′-Azido-2′-deoxy-2′-fluorocytidine-5′-(O-1-naphthyl-N-(S)-1-(3,3-dimethybutoxycarbonyl)ethyl phosphoramidate;
4′-Azido-2′-deoxy-2′-fluorocytidine-5′-(O-1-naphthyl-N-(S)-1-(cyclohexoxycarbonyl)ethyl phosphoramidate;
4′-Azido-2′-deoxy-2′-fluorocytidine-5′-(O-2-naphthyl-N-(S)-1-(isopropoxycarbonyl)ethyl phosphoramidate;
4′-Azido-2′-deoxy-2′-fluorocytidine-5′-{N,N′-bis[(S)-1-(i sopropoxycarbonyl)ethyl]phosphorodiamidate};
4′-Azido-2′-deoxy-2′-fluorocytidine-5′-{N,N′-bis[(S)-1-(2,2-dimethylpropoxycarbonyl)ethyl]phosphorodiamidate};
4′-Azido-2′-deoxy-2′-fluorocytidine-5′-{N,N′-bis[(S)-1-(hexoxycarbonyl)ethyl]phosphorodiamidate};
4′-Azido-2′-deoxy-2′-fluorocytidine-5′-{N,N′-bis[(S)-1-(cyclohexoxycarbonyl)ethyl]phosphorodiamidate};
4′-Azido-2′-deoxy-2′-fluoro-5′-O-(2-oxido-4-H-1,3,2-benzodioxaphosphorin-2-yl)-cytidine;
4′-Azido-2′-deoxy-2′-fluoro-5′-O-[bis(4-methoxyphenoxy)phosphinyl]cytidine;
4′-Azido-2′-deoxy-2′-fluorocytidine-3′,5′-cyclic phosphoric acid isopropyl ester;
4′-Azido-2′-deoxy-2′-fluoroadenosine;
4′-Azidoadenosine;
4′-Azido-2′-deoxy-2′-fluoroadenosine-5′-(O-1-naphthyl-N-(S)-1-(isopropoxycarbonyl)ethyl phosphoramidate;
4′-Azidoadenosine-5′-(O-1-naphthyl-N-(S)-1-(isopropoxycarbonyl)ethyl phosphoramidate;
4′-Azido-2′-deoxy-2′-fluoroadenosine-5′-(O-2-naphthyl-N-(S)-1-(isopropoxycarbonyl)ethyl phosphoramidate;
4′-Azido-2′-deoxy-2′-fluoroadenosine-5′-{N,N′-bis[(S)-1-(i sopropoxycarbonyl)ethyl]phosphorodiamidate;
4′-Azido-2′-deoxy-2′-fluoro-5′-O-(2-oxido-4-H-1,3,2-benzodioxaphosphorin-2-yl)-adenosine;
4′-Azido-2′-deoxy-2′-fluoro-5′-O-[bis(4-methoxyphenoxy)phosphinyl]adenosine;
4′-Azido-2′-deoxy-2′-fluoroadenosine-3′,5′-cyclic phosphoric acid isopropyl ester;
4′-Azido-2′-deoxy-2′-fluoroguanosine;
4′-Azidoguanosine;
4′-Azido-2′-deoxy-2′-fluoroguanosine-5′-(O-phenyl-N-(S)-1-(isopropoxycarbonyl)ethyl phosphoramidate;
4′-Azidoguanosine-5′-(O-phenyl-N-(S)-1-(isopropoxycarbonyl)ethyl phosphoramidate;
4′-Azido-2′-deoxy-2′-fluoroguanosine-5′-(O-1-naphthyl-N-(S)-1-(isopropoxycarbonyl)ethyl phosphoramidate;
4′-Azidoguanosine-5′-(O-1-naphthyl-N-(S)-1-(isopropoxycarbonyl)ethyl phosphoramidate;
4′-Azido-2′-deoxy-2′-fluoroguanosine-5′-(O-1-naphthyl-N-(S)-1-(hexoxycarbonyl)ethyl phosphoramidate;
4′-Azido-2′-deoxy-2′-fluoroguanosine-5′-(O-1-naphthyl-N-(S)-1-(isobutoxycarbonyl)ethyl phosphoramidate;
4′-Azido-2′-deoxy-2′-fluoroguanosine-5′-(O-1-naphthyl-N-(S)-1-(benzyloxycarbonyl)ethyl phosphoramidate;
3′-Acetyl-4′-azido-2′-deoxy-2′-fluoroguanosine-5′-(O-1-naphthyl-N-(S)-1-(isobutoxycarbonyl)ethyl phosphoramidate;
3′-Acetyl-4′-azido-2′-deoxy-2′-fluoroguanosine-5′-(O-1-naphthyl-N-(S)-1-(isopropoxycarbonyl)ethyl phosphoramidate;
3′-Acetyl-4′-azido-2′-deoxy-2′-fluoroguanosine-5′-(O-2-naphthyl-N-(S)-1-(isopropoxycarbonyl)ethyl phosphoramidate;
4′-Azido-2′-deoxy-2′-fluoroguanosine-5′-{N,N′-bis[(S)-1-(i sopropoxycarbonyl)ethyl]phosphorodiamidate;
4′-Azido-2′-deoxy-2′-fluoroguanosine-5′-{N,N′-bis[(S)-1-(isobutoxycarbonyl)ethyl]phosphorodiamidate;
4′-Azido-2′-deoxy-2′-fluoroguanosine-5′-{N,N′-bis[(S)-1-(cyclohexoxycarbonyl)ethyl]phosphorodiamidate;
4′-Azido-2′-deoxy-2′-fluoroguanosine-5′-{N,N′-bis[(S)-1-(cyclopentoxycarbonyl)ethyl]phosphorodiamidate;
4′-Azido-2′-deoxy-2′-fluoroguanosine-5′-{N,N′-bis[(S)-1-(benzyloxycarbonyl)ethyl]phosphorodiamidate;
4′-Azido-2′-deoxy-2′fluoro-5′-O-(2-oxido-4-H-1,3,2-benzodioxaphosphorin-2-yl)-guanosine;
4′-Azido-2′-deoxy-2′-fluoro-5′-O-[bis(4-methoxyphenoxy)phosphinyl]guanosine;
3′-Acetyl-4′-azido-2′-deoxy-2′-fluoro-5′-O-[bis(4-methoxyphenoxy)phosphinyl]guanosine; and
4′-Azido-2′-deoxy-2′-fluoroguanosine-3′,5′-cyclic phosphoric acid isopropyl ester.

The application provides the above method, wherein the Filovirus is Ebolavirus.

The application provides a method for treating an infection with a Filovirus related negative strand RNA virus from the Bornaviridae, Rhabdoviridae, Paramyxoviridae, Arenaviridae, Bunyaviridae or Orthomyxoviridae families, comprising administering to a patient in need thereof a therapeutically effective amount of a compound of Formula I.

The application provides a method for treating an infection with a Filovirus related positive strand RNA virus from the Coronaviridae, Picornaviridae, Flaviviridae or Togaviridae families, comprising administering to a patient in need thereof a therapeutically effective amount of a compound of Formula I.

The application provides any of the above methods, further comprising administering an immune system modulator or an antiviral agent that inhibits viral replication, or a combination of one or more immune system modulators or antiviral agents.

The application provides the above method, wherein the immune system modulator is a TLR agonist, an interferon or chemically derivatized interferon or a vaccine.

The application provides a method for inhibiting replication of a Filovirus in a cell comprising administering to the cell a compound of Formula I.

The application provides a compound of Formula II,

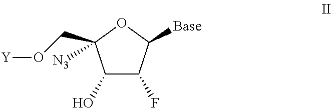

wherein:
Base is adenine;

### Y is P(═X)(R′)(R);

R is O—R¹ or NHR^{1′};
R^{1′} is —C(R^{2a})(R^{2b})C(═O)OR³;

R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
   each R^{1a} is independently H or lower alkyl;
   each R^{1b} is independently —OR^{1a} or —N(R^{1a})₂;
   each R^{1c} is lower alkyl;
   each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
      m is 0, 1, or 2;
      n is 1, 2, or 3;
      p is 1 or 2;
      r is 1 or 2;
   or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and
each R⁴ is independently H, lower alkyl;
or R^{2b} and R⁴ together form (CH₂)₃;
   with the proviso that the compound is not isopropyl ((((2R,3R,4R,5R)-5-(6-amino-9H-purin-9-yl)-2-azido-4-fluoro-3-hydroxytetrahydrofuran-2-yl)methoxy)(phenoxy)phosphoryl)-L-alaninate;
   or a pharmacologically acceptable salt thereof.

The application provides a compound of Formula II,

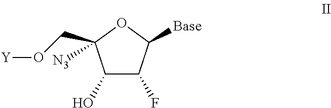

wherein:
Base is cytosine;

### Y is P(═X)(R′)(R);

R is O—R¹ or NHR^{1′};
R^{1′} is —C(R^{2a})(R^{2b})C(═O)OR³;

R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
   each R^{1a} is independently H or lower alkyl;
   each R^{1b} is independently —OR^{1a} or —N(R^{1a})₂;
   each R^{1c} is lower alkyl;
   each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
      m is 0, 1, or 2;
      n is 1, 2, or 3;
      p is 1 or 2;
      r is 1 or 2;
   or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and
each R⁴ is independently H, lower alkyl;
or R^{2b} and R⁴ together form (CH₂)₃;
   with the proviso that the compound is not isopropyl ((((2S,3S,4S,5S)-5-(4-amino-2-oxopyrimidin-1(2H)-yl)-2-azido-4-fluoro-3-hydroxytetrahydrofuran-2-yl)methoxy)(((R)-1-isopropoxy-1-oxopropan-2-yl)amino)phosphoryl)-L-alaninate or isopropyl ((((2R,3R,4R,5R)-5-(4-amino-2-oxopyrimidin-1 (2H)-yl)-2-azido-4-fluoro-3-hydroxytetrahydrofuran-2-yl)methoxy)(phenoxy)phosphoryl)-L-alaninate;
   or a pharmacologically acceptable salt thereof.

The application provides a compound of Formula II,

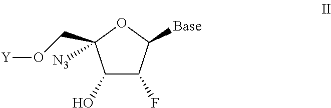

wherein:
Base is uracil;

### Y is P(═X)(R′)(R);

R is O—R¹ or NHR^{1′};
R^{1′} is —C(R^{2a})(R^{2b})C(═O)OR³;

R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
   each R^{1a} is independently H or lower alkyl;
   each R^{1b} is independently —OR^{1a} or —N(R^{1a})₂;
   each R^{1c} is lower alkyl;
   each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
      m is 0, 1, or 2;
      n is 1, 2, or 3;
      p is 1 or 2;
      r is 1 or 2;
   or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and each R⁴ is independently H, lower alkyl;
or R^{2b} and R⁴ together form (CH₂)₃;
   with the proviso that the compound is not isopropyl ((((2S,3S,4S,5S)-2-azido-5-(2,4-dioxo-3,4-dihydropyrimidin-1 (2H)-yl)-4-fluoro-3-hydroxytetrahydrofuran-2-yl)methoxy)(((R)-1-isopropoxy-1-oxopropan-2-yl)amino)phosphoryl)-L-alaninate, isopropyl ((((2R,3R,4S,5R)-2-azido-5-(2,4-dioxo-3,4-dihydropyrimidin-1(2H)-yl)-4-fluoro-3-hydroxytetrahydrofuran-2-yl)methoxy)(phenoxy)phosphoryl)-L-alaninate, or isopropyl ((((2R,3R,4R,5R)-2-azido-5-(2,4-dioxo-3,4-dihydropyrimidin-1(2H)-yl)-4-fluoro-3-hydroxytetrahydrofuran-2-yl)methoxy)(phenoxy)phosphoryl)-L-alaninate;
   or a pharmacologically acceptable salt thereof.

The application provides a compound of Formula II,

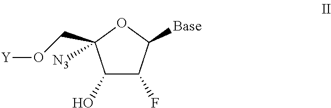

wherein:
Base is thymine;

### Y is P(═X)(R′)(R);

R is O—R¹ or NHR^{1′};
R^{1′} is —C(R^{2a})(R^{2b})C(═O)OR³;

R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
   each R^{1a} is independently H or lower alkyl;
   each R^{1b} is independently —OR^{1a} or —N(R^{1a})₂;
   each R^{1c} is lower alkyl;
   each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
      m is 0, 1, or 2;
      n is 1, 2, or 3;
      p is 1 or 2;
      r is 1 or 2;
   or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and
each R⁴ is independently H, lower alkyl;
or R^{2b} and R⁴ together form (CH₂)₃;
   or a pharmacologically acceptable salt thereof.

The application provides a compound of Formula II selected from the list consisting of:
4′-Azido-2′-deoxy-2′-fluorouridine-5′-(O-1-naphthyl-N-(S)-1-(isopropoxycarbonyl)ethyl phosphoramidate;
4′-Azido-2′-deoxy-2′-fluorouridine-5′-(O-1-naphthyl-N-(S)-1-(i2,2-dimethylpropoxycarbonyl)ethyl phosphoramidate;
4′-Azido-2′-deoxy-2′-fluorouridine-5′-(O-1-naphthyl-N-(S)-1-(benzyloxycarbonyl)ethyl phosphoramidate;
4′-Azido-2′-deoxy-2′-fluorouridine-5′-(O-1-phenyl-N-(S)-1-(hexoxycarbonyl)ethyl phosphoramidate;
4′-Azido-2′-deoxy-2′-fluorouridine-5′-(O-1-naphthyl-N-(S)-1-(cyclopentoxycarbonyl)ethyl phosphoramidate;
4′-Azido-2′-deoxy-2′-fluorouridine-5′-{N,N′-bis[(S)-1-(benzyloxycarbonyl)ethyl]phosphorodiamidate};
4′-Azido-2′-deoxy-2′-fluorouridine-5′-{N,N′-bis[(S)-1-(hexoxycarbonyl)ethyl]phosphorodiamidate};
4′-Azido-2′-deoxy-2′-fluorouridine-5′-{N,N′-bis[(S)-1-(cyclopentoxycarbonyl)ethyl]phosphorodiamidate};
4′-Azido-2′-deoxy-2′-fluorouridine-5′-O-(2-oxido-4-H-1,3,2-benzodioxaphosphorin-2-yl)-uridine;
4′-Azido-2′-deoxy-2′-fluorouridine-5′-O-[bis(4-methoxyphenoxy)phosphinyl]uridine;
4′-Azido-2′-deoxy-2′-fluorocytidine-5′-(O-phenyl-N-(S)-1-(ethoxycarbonyl)ethyl phosphoramidate;
4′-Azido-2′-deoxy-2′-fluorocytidine-5′-(O-phenyl-N-(S)-1-(neopentoxycarbonyl)ethyl phosphoramidate;
4′-Azido-2′-deoxy-2′-fluorocytidine-5′-(O-1-naphthyl-N-(S)-1-(isopropoxycarbonyl)ethyl phosphoramidate;
4′-Azido-2′-deoxy-2′-fluorocytidine-5′-(O-1-naphthyl-N-(S)-1-(2,2-dimethylpropoxycarbonyl)ethyl phosphoramidate;
4′-Azido-2′-deoxy-2′-fluorocytidine-5′-(O-1-naphthyl-N-(S)-1-(benzyloxycarbonyl)ethyl phosphoramidate;
4′-Azido-2′-deoxy-2′-fluorocytidine-5′-(O-1-naphthyl-N-(S)-1-(3,3-dimethybutoxycarbonyl)ethyl phosphoramidate;
4′-Azido-2′-deoxy-2′-fluorocytidine-5′-(O-1-naphthyl-N-(S)-1-(cyclohexoxycarbonyl)ethyl phosphoramidate;
4′-Azido-2′-deoxy-2′-fluorocytidine-5′-(O-2-naphthyl-N-(S)-1-(isopropoxycarbonyl)ethyl phosphoramidate;
4′-Azido-2′-deoxy-2′-fluorocytidine-5′-{N,N′-bis[(S)-1-(2,2-dimethylpropoxycarbonyl)ethyl]phosphorodiamidate};
4′-Azido-2′-deoxy-2′-fluorocytidine-5′-{N,N′-bis[(S)-1-(hexoxycarbonyl)ethyl]phosphorodiamidate};
4′-Azido-2′-deoxy-2′-fluorocytidine-5′-{N,N′-bis[(S)-1-(cyclohexoxycarbonyl)ethyl]phosphorodiamidate};
4′-Azido-2′-deoxy-2′-fluoro-5′-O-(2-oxido-4-H-1,3,2-benzodioxaphosphorin-2-yl)-cytidine;
4′-Azido-2′-deoxy-2′-fluoro-5′-O-[bis(4-methoxyphenoxy)phosphinyl]cytidine;
4′-Azido-2′-deoxy-2′-fluorocytidine-3′,5′-cyclic phosphoric acid isopropyl ester;
4′-Azido-2′-deoxy-2′-fluoroadenosine-5′-(O-1-naphthyl-N-(S)-1-(isopropoxycarbonyl)ethyl phosphoramidate;
4′-Azido-2′-deoxy-2′-fluoroadenosine-5′-(O-2-naphthyl-N-(S)-1-(isopropoxycarbonyl)ethyl phosphoramidate;
4′-Azido-2′-deoxy-2′-fluoroadenosine-5′-{N,N′-bis[(S)-1-(isopropoxycarbonyl)ethyl]phosphorodiamidate;
4′-Azido-2′-deoxy-2′-fluoro-5′-O-(2-oxido-4-H-1,3,2-benzodioxaphosphorin-2-yl)-adenosine;
4′-Azido-2′-deoxy-2′-fluoro-5′-O-[bis(4-methoxyphenoxy)phosphinyl]adenosine;
4′-Azido-2′-deoxy-2′-fluoroadenosine-3′,5′-cyclic phosphoric acid isopropyl ester;
4′-Azido-2′-deoxy-2′-fluoroguanosine-5′-(O-phenyl-N-(S)-1-(isopropoxycarbonyl)ethyl phosphoramidate;
4′-Azido-2′-deoxy-2′-fluoroguanosine-5′-(O-1-naphthyl-N-(S)-1-(isopropoxycarbonyl)ethyl phosphoramidate;
4′-Azido-2′-deoxy-2′-fluoroguanosine-5′-(O-1-naphthyl-N-(S)-1-(hexoxycarbonyl)ethyl phosphoramidate;
4′-Azido-2′-deoxy-2′-fluoroguanosine-5′-(O-1-naphthyl-N-(S)-1-(isobutoxycarbonyl)ethyl phosphoramidate;
4′-Azido-2′-deoxy-2′-fluoroguanosine-5′-(O-1-naphthyl-N-(S)-1-(benzyloxycarbonyl)ethyl phosphoramidate;
3′-Acetyl-4′-azido-2′-deoxy-2′-fluoroguanosine-5′-(O-1-naphthyl-N-(S)-1-(isobutoxycarbonyl)ethyl phosphoramidate;
3′-Acetyl-4′-azido-2′-deoxy-2′-fluoroguanosine-5′-(O-1-naphthyl-N-(S)-1-(isopropoxycarbonyl)ethyl phosphoramidate;
3′-Acetyl-4′-azido-2′-deoxy-2′-fluoroguanosine-5′-(O-2-naphthyl-N-(S)-1-(isopropoxycarbonyl)ethyl phosphoramidate;
4′-Azido-2′-deoxy-2′-fluoroguanosine-5′-{N,N′-bis[(S)-1-(isopropoxycarbonyl)ethyl]phosphorodiamidate;
4′-Azido-2′-deoxy-2′-fluoroguanosine-5′-{N,N′-bis[(S)-1-(isobutoxycarbonyl)ethyl]phosphorodiamidate;
4′-Azido-2′-deoxy-2′-fluoroguanosine-5′-{N,N′-bis[(S)-1-(cyclohexoxycarbonyl)ethyl]phosphorodiamidate;
4′-Azido-2′-deoxy-2′-fluoroguanosine-5′-{N,N′-bis[(S)-1-(cyclopentoxycarbonyl)ethyl]phosphorodiamidate;
4′-Azido-2′-deoxy-2′-fluoroguanosine-5′-{N,N′-bis[(S)-1-(benzyloxycarbonyl)ethyl]phosphorodiamidate;
4′-Azido-2′-deoxy-2′fluoro-5′-O-(2-oxido-4-H-1,3,2-benzodioxaphosphorin-2-yl)-guanosine;
4′-Azido-2′-deoxy-2′-fluoro-5′-O-[bis(4-methoxyphenoxy)phosphinyl]guanosine;
3′-Acetyl-4′-azido-2′-deoxy-2′-fluoro-5′-O-[bis(4-methoxyphenoxy)phosphinyl]guanosine; and
4′-Azido-2′-deoxy-2′-fluoroguanosine-3′,5′-cyclic phosphoric acid isopropyl ester.

The application provides a compound of Formula II selected from the group consisting of:
4′-Azido-2′-deoxy-2′-fluorouridine-5′-(O-phenyl-N-(S)-1-(isopropoxycarbonyl)ethyl phosphoramidate;
4′-Azido-2′-deoxy-2′-fluorouridine-5′-{N,N′-bis[(S)-1-(isopropoxycarbonyl)ethyl]phosphorodiamidate};
4′-Azido-2′-deoxy-2′-fluorocytidine-5′-(O-phenyl-N-(S)-1-(isopropoxycarbonyl)ethyl phosphoramidate; and
4′-Azido-2′-deoxy-2′-fluorocytidine-5′-{N,N′-bis[(S)-1-(isopropoxycarbonyl)ethyl]phosphorodiamidate}.

The application provides a compound of Formula II, wherein the compound is 4′-Azidouridine-5′-(O-1-naphthyl-N-(S)-1-(benzyloxycarbonyl)ethyl phosphoramidate.

The application provides a compound of Formula I, wherein the compound is selected from the group consisting of:
4′-Azidouridine-5′-(O-phenyl-N-(S)-1-(isopropoxycarbonyl)ethyl phosphoramidate;
4′-Azidocytidine-5′-(O-phenyl-N-(S)-1-(benzyloxycarbonyl)ethyl phosphoramidate;
4′-Azidocytidine-5′-(O-phenyl-N-(S)-1-(eisopropoxycarbonyl)ethyl phosphoramidate;
4′-Azidocytidine-5′-(O-naphthyl-N-(S)-1-(benzyloxycarbonyl)ethyl phosphoramidate;
4′-Azidoadenosine-5′-(O-1-naphthyl-N-(S)-1-(isopropoxycarbonyl)ethyl phosphoramidate;
4′-Azidoguanosine-5′-(O-phenyl-N-(S)-1-(isopropoxycarbonyl)ethyl phosphoramidate; and
4′-Azidoguanosine-5′-(O-1-naphthyl-N-(S)-1-(isopropoxycarbonyl)ethyl phosphoramidate.

The application provides a method for treating an infection with a Filovirus related negative strand RNA virus from the Bornaviridae, Rhabdoviridae, Paramyxoviridae, Arenaviridae, Bunyaviridae or Orthomyxoviridae families, comprising administering to a patient in need thereof a therapeutically effective amount of a compound of Formula II.

The application provides a method for treating an infection with a Filovirus related positive strand RNA virus from the Coronaviridae, Picornaviridae, Flaviviridae or Togaviridae families, comprising administering to a patient in need thereof a therapeutically effective amount of a compound of Formula II.

The application provides any of the above methods, further comprising administering an immune system modulator or an antiviral agent that inhibits viral replication, or a combination of one or more immune system modulators or antiviral agents.

The application provides the above method, wherein the immune system modulator is a TLR agonist, an interferon or chemically derivatized interferon or a vaccine.

The application provides a method for inhibiting replication of a Filovirus in a cell comprising administering to the cell a compound of Formula II.

The application provides any of the above methods wherein Base in the compound of Formula II is guanine.

The application provides any of the above methods wherein Base in the compound of Formula II is adenine.

The application provides any of the above methods wherein Base in the compound of Formula II is uracil.

The application provides any of the above methods wherein Base in the compound of Formula II is cytosine.

The application provides any of the above methods wherein Base in the compound of Formula II is thymine.

The application provides a use of the compound of Formula I in the manufacture of a medicament for the treatment, pre-exposure or post-exposure prophylaxis of a Filovirus infection.

The application provides a use of the compound of Formula II in the manufacture of a medicament for the treatment, pre-exposure or post-exposure prophylaxis of a Filovirus infection.

The application provides a compound, composition, or method as described herein.

Examples of representative compounds encompassed by the present invention and within the scope of the invention are provided in the following Tables. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on standard nucleic acid nomenclature common to one of ordinary skill in the art. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE 1
Examples of compounds of generic Formula I.

| Compound | | |
|---|---|---|
| Number | Structure | Name |
| I-1 | 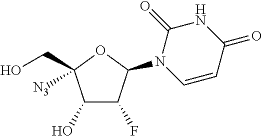 [SMILES: [N-]=[N+]=N[C@]1(CO)O[C@@H](n2ccc(=O)[nH]c2=O)[C@H](F)[C@@H]1O] | 4′-Azido-2′-deoxy-2′- fluorouridine |
| I-2 | 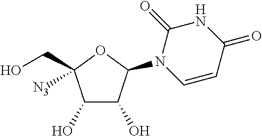 [SMILES: [N-]=[N+]=N[C@]1(CO)O[C@@H](n2ccc(=O)[nH]c2=O)[C@H](O)[C@@H]1O] | 4′-Azidouridine |
| I-3 | 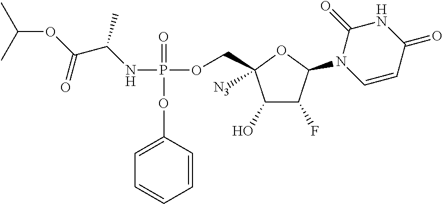 [SMILES: CC(C)OC(=O)[C@H](C)NP(=O)(OC[C@@]1(N=[N+]=[N-])O[C@@H](n2ccc(=O)[nH]c2=O)[C@H](F)[C@@H]1O)Oc1ccccc1] | 4′-Azido-2′-deoxy-2′- fluorouridine-5′-(O- phenyl-N-(S)-1- (isopropoxycarbonyl)ethyl phosphoramidate |
| I-4 | 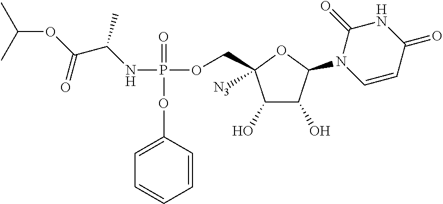 [SMILES: CC(C)OC(=O)[C@H](C)NP(=O)(OC[C@@]1(N=[N+]=[N-])O[C@@H](n2ccc(=O)[nH]c2=O)[C@H](O)[C@@H]1O)Oc1ccccc1] | 4′-Azidouridine-5′-(O- phenyl-N-(S)-1- (isopropoxycarbonyl)ethyl phosphoramidate |
| I-5 | 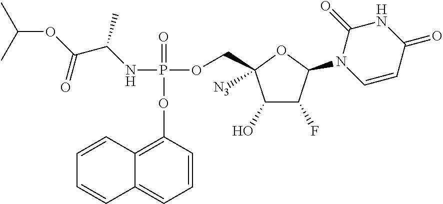 [SMILES: CC(C)OC(=O)[C@H](C)NP(=O)(OC[C@@]1(N=[N+]=[N-])O[C@@H](n2ccc(=O)[nH]c2=O)[C@H](F)[C@@H]1O)Oc1cccc2ccccc12] | 4′-Azido-2′-deoxy-2′- fluorouridine-5′-(O-1- naphthyl-N-(S)-1- (isopropoxycarbonyl)ethyl phosphoramidate |
| I-6 | 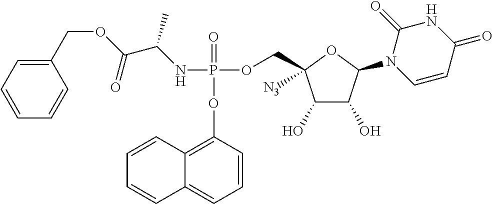 [SMILES: C[C@H](NP(=O)(OC[C@@]1(N=[N+]=[N-])O[C@@H](n2ccc(=O)[nH]c2=O)[C@H](O)[C@@H]1O)Oc1cccc2ccccc12)C(=O)OCc1ccccc1] | 4′-Azidouridine-5′-(O-1- naphthyl-N-(S)-1- (benzyloxycarbonyl)ethyl phosphoramidate |
| I-7 | 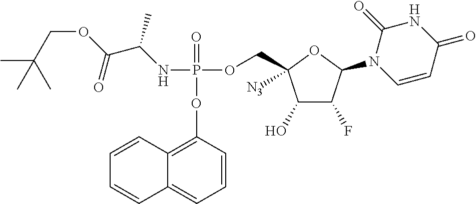 [SMILES: C[C@H](NP(=O)(OC[C@@]1(N=[N+]=[N-])O[C@@H](n2ccc(=O)[nH]c2=O)[C@H](F)[C@@H]1O)Oc1cccc2ccccc12)C(=O)OCC(C)(C)C] | 4′-Azido-2′-deoxy-2′- fluorouridine-5′-(O-1- naphthyl-N-(S)-1-(i2,2- dimethylpropoxycarbonyl) ethyl phosphoramidate |
| I-8 | 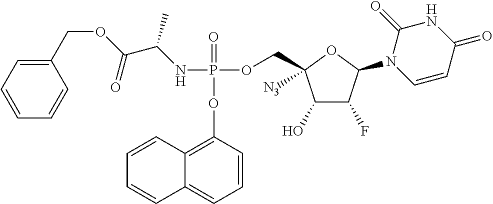 [SMILES: C[C@H](NP(=O)(OC[C@@]1(N=[N+]=[N-])O[C@@H](n2ccc(=O)[nH]c2=O)[C@H](F)[C@@H]1O)Oc1cccc2ccccc12)C(=O)OCc1ccccc1] | 4′-Azido-2′-deoxy-2′- fluorouridine-5′-(O-1- naphthyl-N-(S)-1- (benzyloxycarbonyl)ethyl phosphoramidate |
| I-9 | 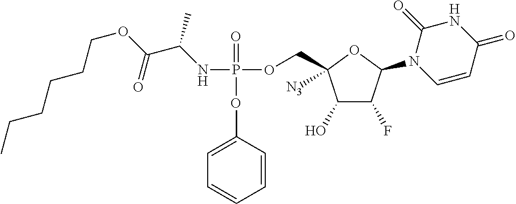 [SMILES: CCCCCCOC(=O)[C@H](C)NP(=O)(OC[C@@]1(N=[N+]=[N-])O[C@@H](n2ccc(=O)[nH]c2=O)[C@H](F)[C@@H]1O)Oc1ccccc1] | 4′-Azido-2′-deoxy-2′- fluorouridine-5′-(O-1- phenyl-N-(S)- 1-(hexoxycarbonyl)ethyl phosphoramidate |
| I-10 | 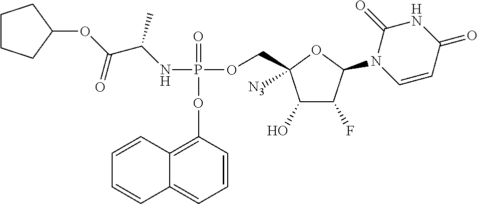 [SMILES: C[C@H](NP(=O)(OC[C@@]1(N=[N+]=[N-])O[C@@H](n2ccc(=O)[nH]c2=O)[C@H](F)[C@@H]1O)Oc1cccc2ccccc12)C(=O)OC1CCCC1] | 4′-Azido-2′-deoxy-2′- fluorouridine-5′-(O-1- naphthyl-N- (S)-1-(cyclopentyl- oxycarbonyl)ethyl phosphoramidate |
| I-11 | 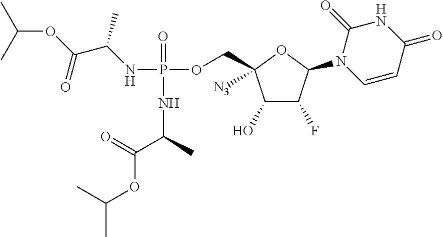 [SMILES: CC(C)OC(=O)[C@H](C)NP(=O)(N[C@@H](C)C(=O)OC(C)C)OC[C@@]1(N=[N+]=[N-])O[C@@H](n2ccc(=O)[nH]c2=O)[C@H](F)[C@@H]1O] | 4′-Azido-2′-deoxy-2′- fluorouridine-5′-{N,N′- bis[(S)-1- (isopropoxycarbonyl)ethyl] phosphorodiamidate} |
| I-12 | 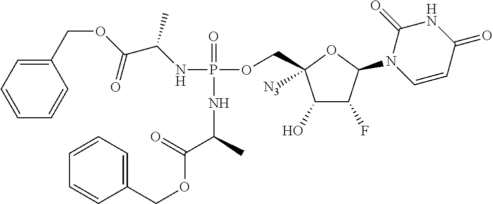 [SMILES: C[C@H](NP(=O)(N[C@@H](C)C(=O)OCc1ccccc1)OC[C@@]1(N=[N+]=[N-])O[C@@H](n2ccc(=O)[nH]c2=O)[C@H](F)[C@@H]1O)C(=O)OCc1ccccc1] | 4′-Azido-2′-deoxy-2′- fluorouridine-5′-{N,N′- bis[(S)1- (benzyloxycarbonyl)ethyl] phosphorodiamidate} |
| I-13 | 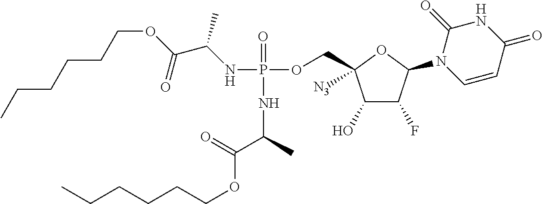 [SMILES: CCCCCCOC(=O)[C@H](C)NP(=O)(N[C@@H](C)C(=O)OCCCCCC)OC[C@@]1(N=[N+]=[N-])O[C@@H](n2ccc(=O)[nH]c2=O)[C@H](F)[C@@H]1O] | 4′-Azido-2′-deoxy-2′- fluorouridine-5′-{N,N′- bis[(S)-1- (hexyoxycarbonyl)ethyl] phosphorodiamidate} |
| I-14 | 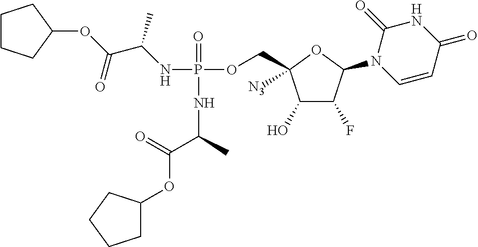 [SMILES: C[C@H](NP(=O)(N[C@@H](C)C(=O)OC1CCCC1)OC[C@@]1(N=[N+]=[N-])O[C@@H](n2ccc(=O)[nH]c2=O)[C@H](F)[C@@H]1O)C(=O)OC1CCCC1] | 4′-Azido-2′-deoxy-2′- fluorouridine-5′-{N,N′- bis[(S)-1-(cyclopentyloxy- carbonyl)ethyl] phosphorodiamidate} |
| I-15 | 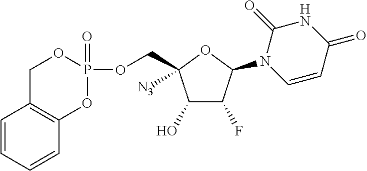 [SMILES: [N-]=[N+]=N[C@]1(COP2(=O)OCc3ccccc3O2)O[C@@H](n2ccc(=O)[nH]c2=O)[C@H](F)[C@@H]1O] | 4′-Azido-2′-deoxy-2′- fluorouridine-5′-O-(2- oxido-4-H-1,3,2- benzodioxaphosphorin- 2-yl)-uridine |
| I-16 | 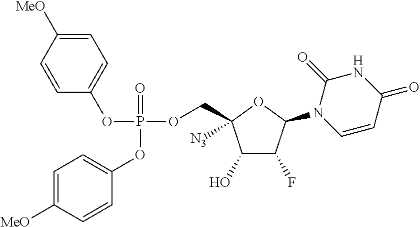 [SMILES: COc1ccc(OP(=O)(OC[C@@]2(N=[N+]=[N-])O[C@@H](n3ccc(=O)[nH]c3=O)[C@H](F)[C@@H]2O)Oc2ccc(OC)cc2)cc1] | 4′-Azido-2′-deoxy-2′- fluorouridine-5′-O-[bis(4- methoxyphenoxy) phosphinyl]uridine |
| I-17 | 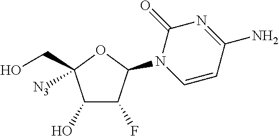 [SMILES: [N-]=[N+]=N[C@]1(CO)O[C@@H](n2ccc(N)nc2=O)[C@H](F)[C@@H]1O] | 4′-Azido-2′-deoxy-2′- fluorocytidine |
| I-18 | 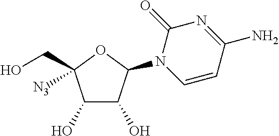 [SMILES: [N-]=[N+]=N[C@]1(CO)O[C@@H](n2ccc(N)nc2=O)[C@H](O)[C@@H]1O] | 4′-Azidocytidine |
| I-19 | 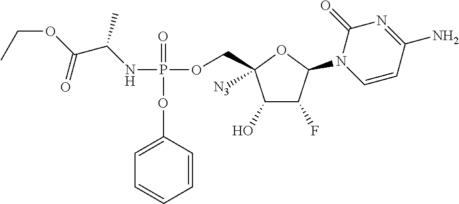 [SMILES: CCOC(=O)[C@H](C)NP(=O)(OC[C@@]1(N=[N+]=[N-])O[C@@H](n2ccc(N)nc2=O)[C@H](F)[C@@H]1O)Oc1ccccc1] | 4′-Azido-2′-deoxy-2′- fluorocytidine-5′-(O- phenyl-N-(S)-1- (ethoxycarbonyl)ethyl phosphoramidate |
| I-20 | 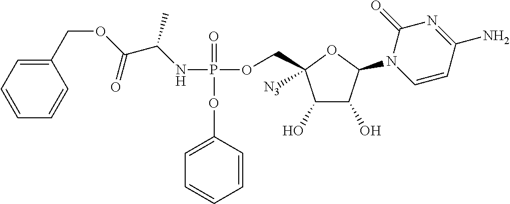 [SMILES: C[C@H](NP(=O)(OC[C@@]1(N=[N+]=[N-])O[C@@H](n2ccc(N)nc2=O)[C@H](O)[C@@H]1O)Oc1ccccc1)C(=O)OCc1ccccc1] | 4′-Azidocytidine-5′- (O-phenyl-N-(S)-1- (benzyloxycarbonyl)ethyl phosphoramidate |
| I-21 | 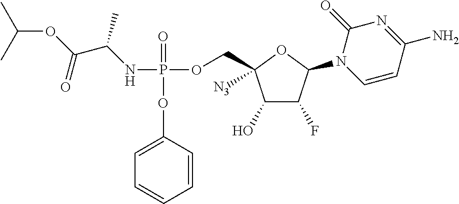 [SMILES: CC(C)OC(=O)[C@H](C)NP(=O)(OC[C@@]1(N=[N+]=[N-])O[C@@H](n2ccc(N)nc2=O)[C@H](F)[C@@H]1O)Oc1ccccc1] | 4′-Azido-2′-deoxy-2′- fluorocytidine-5′-(O- phenyl-N-(S)-1- (isopropoxycarbonyl)ethyl phosphoramidate |
| I-22 | 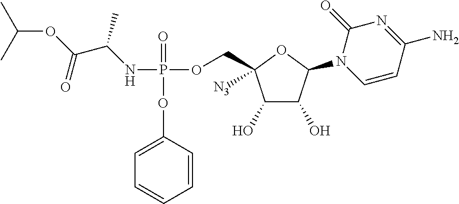 [SMILES: CC(C)OC(=O)[C@H](C)NP(=O)(OC[C@@]1(N=[N+]=[N-])O[C@@H](n2ccc(N)nc2=O)[C@H](O)[C@@H]1O)Oc1ccccc1] | 4′-Azidocytidine-5′- (O-phenyl-N-(S)-1- (eisopropoxycarbonyl)ethyl phosphoramidate |
| I-23 | 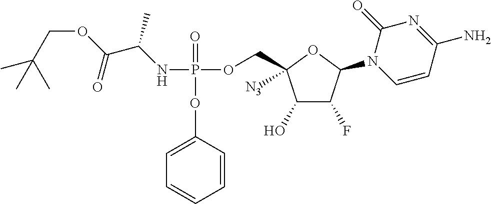 [SMILES: C[C@H](NP(=O)(OC[C@@]1(N=[N+]=[N-])O[C@@H](n2ccc(N)nc2=O)[C@H](F)[C@@H]1O)Oc1ccccc1)C(=O)OCC(C)(C)C] | 4′-Azido-2′-deoxy-2′- fluorocytidine-5′-(O- phenyl-N-(S)-1- (neopentoxycarbonyl)ethyl phosphoramidate |
| I-24 | 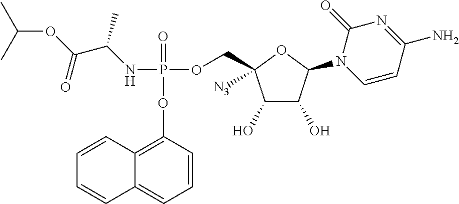 [SMILES: CC(C)OC(=O)[C@H](C)NP(=O)(OC[C@@]1(N=[N+]=[N-])O[C@@H](n2ccc(N)nc2=O)[C@H](O)[C@@H]1O)Oc1cccc2ccccc12] | 4′-Azido-2′-deoxy-2′- fluorocytidine-5′-(O-1- naphthyl-N-(S)-1- (isopropoxycarbonyl)ethyl phosphoramidate |
| I-25 | 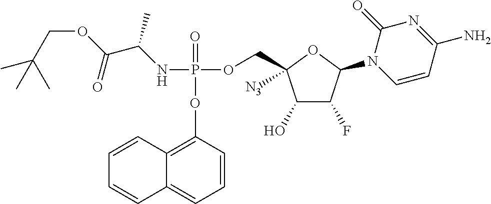 [SMILES: C[C@H](NP(=O)(OC[C@@]1(N=[N+]=[N-])O[C@@H](n2ccc(N)nc2=O)[C@H](F)[C@@H]1O)Oc1cccc2ccccc12)C(=O)OCC(C)(C)C] | 4′-Azido-2′-deoxy-2′- fluorocytidine-5′-(O-1- naphthyl-N-(S)-1-(2,2- dimethylpropoxy- carbonyl)ethyl phosphoramidate |
| I-26 | 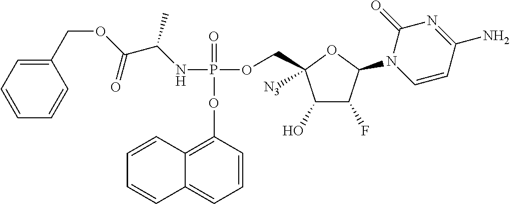 [SMILES: C[C@H](NP(=O)(OC[C@@]1(N=[N+]=[N-])O[C@@H](n2ccc(N)nc2=O)[C@H](F)[C@@H]1O)Oc1cccc2ccccc12)C(=O)OCc1ccccc1] | 4′-Azido-2′-deoxy-2′- fluorocytidine-5′-(O-1- naphthyl-N-(S)-1- (benzyloxycarbonyl)ethyl phosphoramidate |
| I-27 | 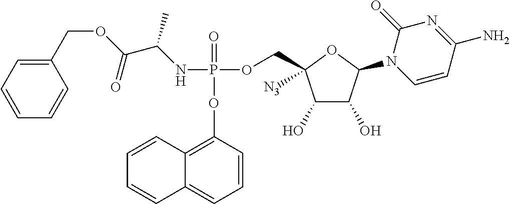 [SMILES: C[C@H](NP(=O)(OC[C@@]1(N=[N+]=[N-])O[C@@H](n2ccc(N)nc2=O)[C@H](O)[C@@H]1O)Oc1cccc2ccccc12)C(=O)OCc1ccccc1] | 4′-Azidocytidine-5′-(O- naphthyl-N-(S)-1- (benzyloxycarbonyl)ethyl phosphoramidate |
| I-28 | 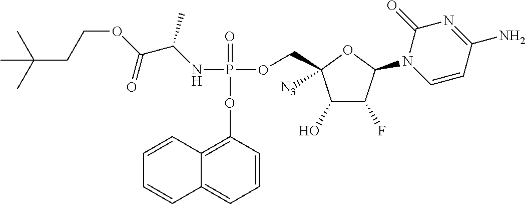 [SMILES: C[C@H](NP(=O)(OC[C@@]1(N=[N+]=[N-])O[C@@H](n2ccc(N)nc2=O)[C@H](F)[C@@H]1O)Oc1cccc2ccccc12)C(=O)OCCC(C)(C)C] | 4′-Azido-2′-deoxy-2′- fluorocytidine-5′-(O-1- naphthyl-N-(S)-1-(3,3- dimethybutoxy- carbonyl)ethyl phosphoramidate |
| I-29 | 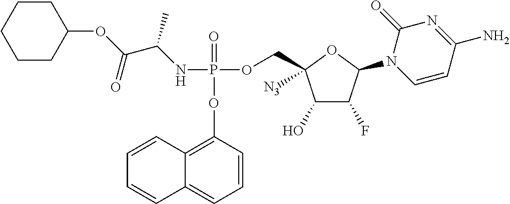 [SMILES: C[C@H](NP(=O)(OC[C@@]1(N=[N+]=[N-])O[C@@H](n2ccc(N)nc2=O)[C@H](F)[C@@H]1O)Oc1cccc2ccccc12)C(=O)OC1CCCCC1] | 4′-Azido-2′-deoxy-2′- fluorocytidine-5′-(O-1- naphthyl-N-(S)-1- (cyclohexoxy- carbonyl)ethyl phosphoramidate |
| I-30 | 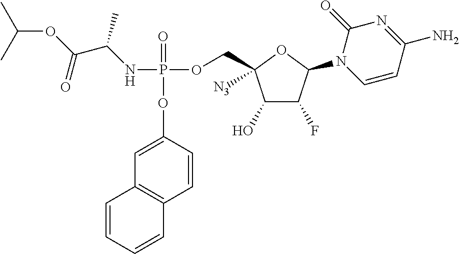 [SMILES: CC(C)OC(=O)[C@H](C)NP(=O)(OC[C@@]1(N=[N+]=[N-])O[C@@H](n2ccc(N)nc2=O)[C@H](F)[C@@H]1O)Oc1ccc2ccccc2c1] | 4′-Azido-2′-deoxy-2′- fluorocytidine-5′-(O-2- naphthyl-N-(S)-1- (isopropoxy- carbonyl)ethyl phosphoramidate |
| I-31 | 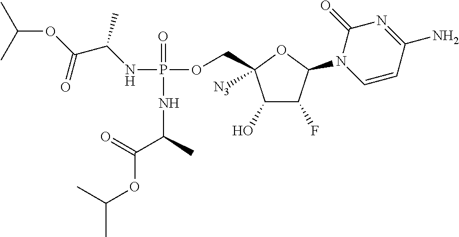 [SMILES: CC(C)OC(=O)[C@H](C)NP(=O)(N[C@@H](C)C(=O)OC(C)C)OC[C@@]1(N=[N+]=[N-])O[C@@H](n2ccc(N)nc2=O)[C@H](F)[C@@H]1O] | 4′-Azido-2′-deoxy-2′- fluorocytidine-5′-{N,N′- bis[(S)-1-(isopro- poxycarbonyl)ethyl] phosphorodiamidate} |
| I-32 | 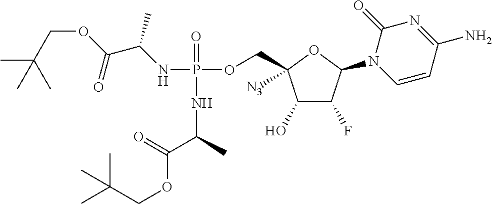 [SMILES: C[C@H](NP(=O)(N[C@@H](C)C(=O)OCC(C)(C)C)OC[C@@]1(N=[N+]=[N-])O[C@@H](n2ccc(N)nc2=O)[C@H](F)[C@@H]1O)C(=O)OCC(C)(C)C] | 4′-Azido-2′-deoxy-2′- fluorocytidine-5′-{N,N- bis[(S)-1-(2,2- dimethylpropoxy- carbonyl)ethyl] phosphorodiamidate} |
| I-33 | 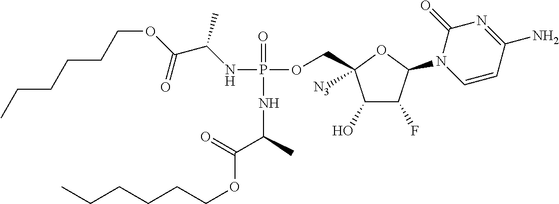 [SMILES: CCCCCCOC(=O)[C@H](C)NP(=O)(N[C@@H](C)C(=O)OCCCCCC)OC[C@@]1(N=[N+]=[N-])O[C@@H](n2ccc(N)nc2=O)[C@H](F)[C@@H]1O] | 4′-Azido-2′-deoxy-2′- fluorocytidine-5′-{N,N′- bis[(S)-1- (hexoxycarbonyl)ethyl] phosphorodiamidate} |
| I-34 | 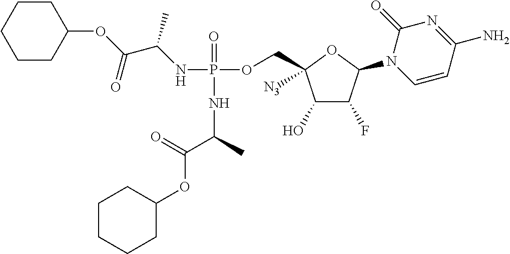 [SMILES: C[C@H](NP(=O)(N[C@@H](C)C(=O)OC1CCCCC1)OC[C@@]1(N=[N+]=[N-])O[C@@H](n2ccc(N)nc2=O)[C@H](F)[C@@H]1O)C(=O)OC1CCCCC1] | 4′-Azido-2′-deoxy-2′- fluorocytidine-5′-{N,N- bis[(S)-1-(cyclo- hexoxycarbonyl)ethyl] phosphorodiamidate} |
| I-35 | 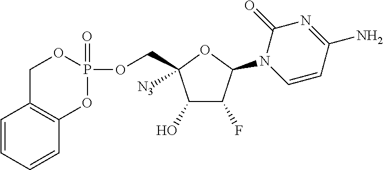 [SMILES: [N-]=[N+]=N[C@]1(COP2(=O)OCc3ccccc3O2)O[C@@H](n2ccc(N)nc2=O)[C@H](F)[C@@H]1O] | 4′-Azido-2′-deoxy- 2′-fluoro-5′-O-(2- oxido-4-H-1,3,2- benzodioxaphosphorin- 2-yl)-cytidine |
| I-36 | 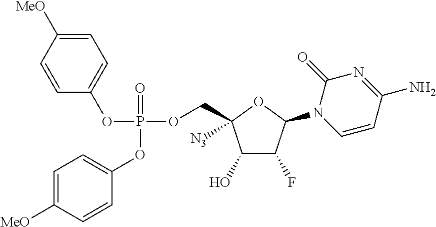 [SMILES: COc1ccc(OP(=O)(OC[C@@]2(N=[N+]=[N-])O[C@@H](n3ccc(N)nc3=O)[C@H](F)[C@@H]2O)Oc2ccc(OC)cc2)cc1] | 4′-Azido-2′-deoxy-2′- fluoro-5′-O-[bis(4- methoxyphenoxy) phosphinyl]cytidine |
| I-37 | 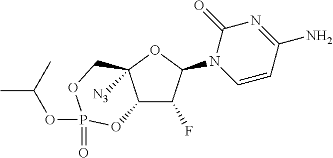 | 4′-Azido-2′-deoxy-2′- fluorocytidine-3′,5′- cyclic phosphoric acid isopropyl ester |
| I-38 | 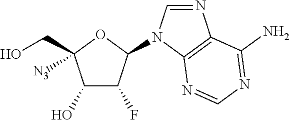 [SMILES: [N-]=[N+]=N[C@]1(CO)O[C@@H](n2cnc3c(N)ncnc32)[C@H](F)[C@@H]1O] | 4′-Azido-2′-deoxy-2′- fluoroadenosine |
| I-39 | 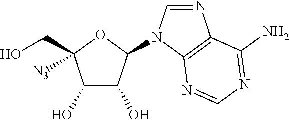 [SMILES: [N-]=[N+]=N[C@]1(CO)O[C@@H](n2cnc3c(N)ncnc32)[C@H](O)[C@@H]1O] | 4′-Azidoadenosine |
| I-40 | 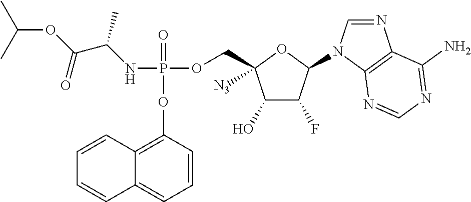 [SMILES: CC(C)OC(=O)[C@H](C)NP(=O)(OC[C@@]1(N=[N+]=[N-])O[C@@H](n2cnc3c(N)ncnc32)[C@H](F)[C@@H]1O)Oc1cccc2ccccc12] | 4′-Azido-2′-deoxy-2′- fluoroadenosine-5′-(O-1- naphthyl-N-(S)-1- (isopropoxycarbonyl)ethyl phosphoramidate |
| I-41 | 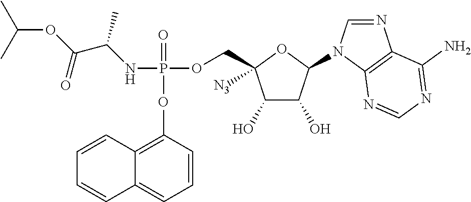 [SMILES: CC(C)OC(=O)[C@H](C)NP(=O)(OC[C@@]1(N=[N+]=[N-])O[C@@H](n2cnc3c(N)ncnc32)[C@H](O)[C@@H]1O)Oc1cccc2ccccc12] | 4′-Azidoadenosine-5′-(O- 1-naphthyl-N-(S)-1- (isopropoxycarbonyl)ethyl phosphoramidate |
| I-42 | 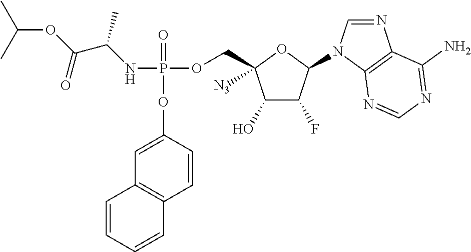 [SMILES: CC(C)OC(=O)[C@H](C)NP(=O)(OC[C@@]1(N=[N+]=[N-])O[C@@H](n2cnc3c(N)ncnc32)[C@H](F)[C@@H]1O)Oc1ccc2ccccc2c1] | 4′-Azido-2′-deoxy-2′- fluoroadenosine-5′-(O-2- naphthyl-N-(S)-1- (isopropoxycarbonyl)ethyl phosphoramidate |
| I-43 | 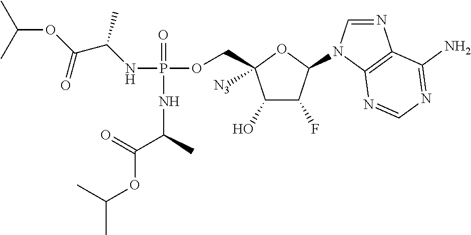 [SMILES: CC(C)OC(=O)[C@H](C)NP(=O)(N[C@@H](C)C(=O)OC(C)C)OC[C@@]1(N=[N+]=[N-])O[C@@H](n2cnc3c(N)ncnc32)[C@H](F)[C@@H]1O] | 4′-Azido-2′-deoxy-2′- fluoroadenosine-5′-{N,N- bis[(S)-1- (isopropoxycarbonyl)ethyl] phosphorodiamidate |
| I-44 | 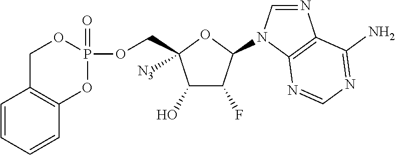 [SMILES: [N-]=[N+]=N[C@]1(COP2(=O)OCc3ccccc3O2)O[C@@H](n2cnc3c(N)ncnc32)[C@H](F)[C@@H]1O] | 4′-Azido-2′-deoxy- 2′-fluoro-5′-O- (2-oxido-4-H-1,3,2- benzodioxaphosphorin-2- yl)-adenosine |
| I-45 | 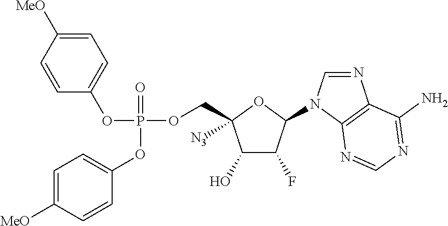 [SMILES: COc1ccc(OP(=O)(OC[C@@]2(N=[N+]=[N-])O[C@@H](n3cnc4c(N)ncnc43)[C@H](F)[C@@H]2O)Oc2ccc(OC)cc2)cc1] | 4′-Azido-2′-deoxy-2′- fluoro-5′-O-[bis(4- methoxyphenoxy) phosphinyl]adenosine |
| I-46 | 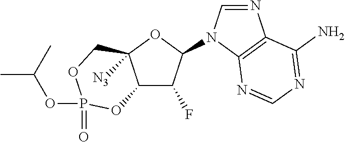 | 4′-Azido-2′-deoxy-2′- fluoroadenosine-3′,5′- cyclic phosphoric acid isopropyl ester |
| I-47 | 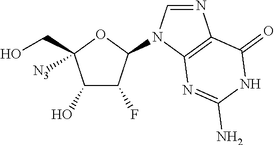 [SMILES: [N-]=[N+]=N[C@]1(CO)O[C@@H](n2cnc3c(=O)[nH]c(N)nc32)[C@H](F)[C@@H]1O] | 4′-Azido-2′-deoxy-2′- fluoroguanosine |
| I-48 | 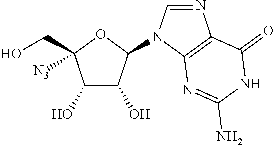 [SMILES: [N-]=[N+]=N[C@]1(CO)O[C@@H](n2cnc3c(=O)[nH]c(N)nc32)[C@H](O)[C@@H]1O] | 4′-Azidoguanosine |
| I-49 | 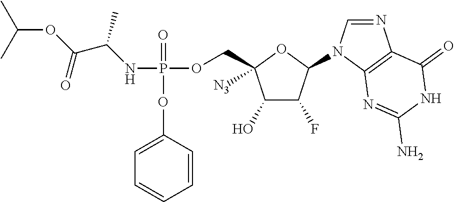 [SMILES: CC(C)OC(=O)[C@H](C)NP(=O)(OC[C@@]1(N=[N+]=[N-])O[C@@H](n2cnc3c(=O)[nH]c(N)nc32)[C@H](F)[C@@H]1O)Oc1ccccc1] | 4′-Azido-2′-deoxy-2′- fluoroguanosine-5′-(O- phenyl-N-(S)-1- (isopropoxycarbonyl)ethyl phosphoramidate |
| I-50 | 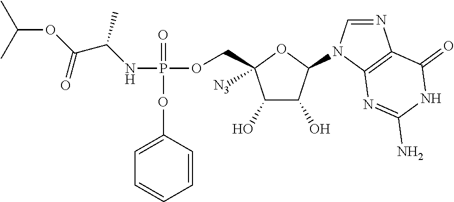 [SMILES: CC(C)OC(=O)[C@H](C)NP(=O)(OC[C@@]1(N=[N+]=[N-])O[C@@H](n2cnc3c(=O)[nH]c(N)nc32)[C@H](O)[C@@H]1O)Oc1ccccc1] | 4′-Azidoguanosine-5′-(O- phenyl-N-(S)-1- (isopropoxycarbonyl)ethyl phosphoramidate |
| I-51 | 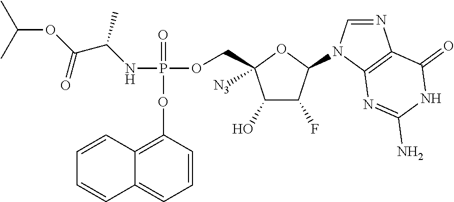 [SMILES: CC(C)OC(=O)[C@H](C)NP(=O)(OC[C@@]1(N=[N+]=[N-])O[C@@H](n2cnc3c(=O)[nH]c(N)nc32)[C@H](F)[C@@H]1O)Oc1cccc2ccccc12] | 4′-Azido-2′-deoxy-2′- fluoroguanosine-5′-(O-1- naphthyl-N-(S)-1- (isopropoxycarbonyl)ethyl phosphoramidate |
| I-52 | 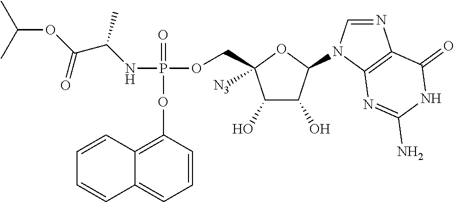 [SMILES: CC(C)OC(=O)[C@H](C)NP(=O)(OC[C@@]1(N=[N+]=[N-])O[C@@H](n2cnc3c(=O)[nH]c(N)nc32)[C@H](O)[C@@H]1O)Oc1cccc2ccccc12] | 4′-Azidoguanosine-5′-(O-1- naphthyl-N-(S)-1- (isopropoxycarbonyl)ethyl phosphoramidate |
| I-53 | 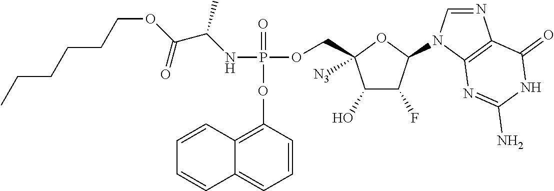 [SMILES: CCCCCCOC(=O)[C@H](C)NP(=O)(OC[C@@]1(N=[N+]=[N-])O[C@@H](n2cnc3c(=O)[nH]c(N)nc32)[C@H](F)[C@@H]1O)Oc1cccc2ccccc12] | 4′-Azido-2′-deoxy-2′- fluoroguanosine-5′-(O-1- naphthyl-N-(S)-1- (hexoxycarbonyl)ethyl phosphoramidate |
| I-54 | 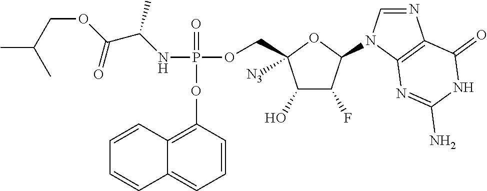 [SMILES: CC(C)COC(=O)[C@H](C)NP(=O)(OC[C@@]1(N=[N+]=[N-])O[C@@H](n2cnc3c(=O)[nH]c(N)nc32)[C@H](F)[C@@H]1O)Oc1cccc2ccccc12] | 4′-Azido-2′-deoxy-2′- fluoroguanosine-5′-(O-1- naphthyl-N-(S)-1- (isobutoxycarbonyl)ethyl phosphoramidate |
| I-55 | 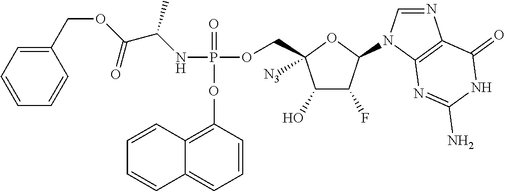 [SMILES: C[C@H](NP(=O)(OC[C@@]1(N=[N+]=[N-])O[C@@H](n2cnc3c(=O)[nH]c(N)nc32)[C@H](F)[C@@H]1O)Oc1cccc2ccccc12)C(=O)OCc1ccccc1] | 4′-Azido-2′-deoxy-2′- fluoroguanosine-5′-(O-1- naphthyl-N-(S)-1- (benzyloxycarbonyl)ethyl phosphoramidate |
| I-56 | 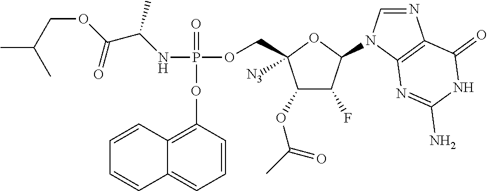 [SMILES: CC(=O)O[C@H]1[C@@H](F)[C@H](n2cnc3c(=O)[nH]c(N)nc32)O[C@@]1(COP(=O)(N[C@@H](C)C(=O)OCC(C)C)Oc1cccc2ccccc12)N=[N+]=[N-]] | 3′-Acetyl-4′-azido-2′- deoxy-2′- fluoroguanosine-5′-(O-1- naphthyl-N-(S)-1- (isobutoxycarbonyl)ethyl phosphoramidate |
| I-57 | 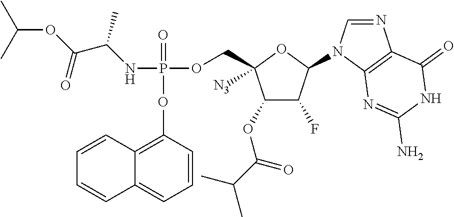 [SMILES: CC(C)OC(=O)[C@H](C)NP(=O)(OC[C@@]1(N=[N+]=[N-])O[C@@H](n2cnc3c(=O)[nH]c(N)nc32)[C@H](F)[C@@H]1OC(=O)C(C)C)Oc1cccc2ccccc12] | 3′-Acetyl-4′-azido-2′- deoxy-2′- fluoroguanosine-5′-(O-1- naphthyl-N-(S)-1- (isopropoxycarbonyl)ethyl phosphoramidate |
| I-58 | 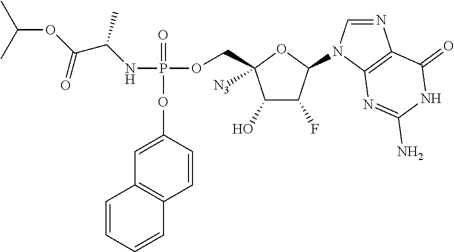 [SMILES: CC(C)OC(=O)[C@H](C)NP(=O)(OC[C@@]1(N=[N+]=[N-])O[C@@H](n2cnc3c(=O)[nH]c(N)nc32)[C@H](F)[C@@H]1O)Oc1ccc2ccccc2c1] | 3′-Acetyl-4′-azido- 2′-deoxy-2′- fluoroguanosine-5′- (O-2-naphthyl-N-(S)-1- (isopropoxycarbonyl)ethyl phosphoramidate |
| I-59 | 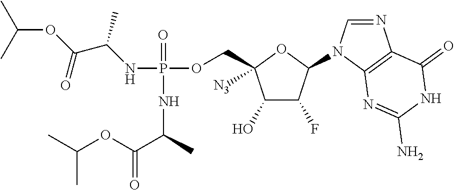 [SMILES: CC(C)OC(=O)[C@H](C)NP(=O)(N[C@@H](C)C(=O)OC(C)C)OC[C@@]1(N=[N+]=[N-])O[C@@H](n2cnc3c(=O)[nH]c(N)nc32)[C@H](F)[C@@H]1O] | 4′-Azido-2′-deoxy-2′- fluoroguanosine-5′-{N,N′- bis[(S)-1- (isopropoxycarbonyl)ethyl] phosphorodiamidate |
| I-60 | 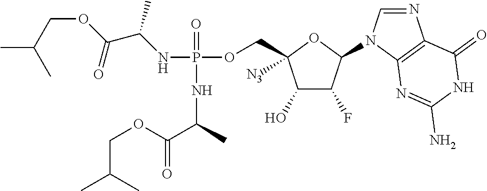 [SMILES: CC(C)COC(=O)[C@H](C)NP(=O)(N[C@@H](C)C(=O)OCC(C)C)OC[C@@]1(N=[N+]=[N-])O[C@@H](n2cnc3c(=O)[nH]c(N)nc32)[C@H](F)[C@@H]1O] | 4′-Azido-2′-deoxy-2′- fluoroguanosine-5′-{N,N′- bis[(S)-1- (isobutoxycarbonyl)ethyl] phosphorodiamidate |
| I-61 | 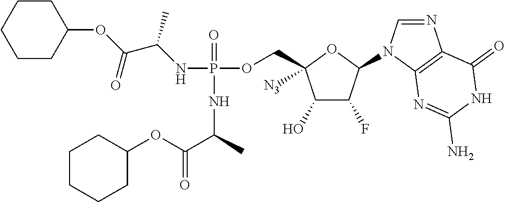 [SMILES: C[C@H](NP(=O)(N[C@@H](C)C(=O)OC1CCCCC1)OC[C@@]1(N=[N+]=[N-])O[C@@H](n2cnc3c(=O)[nH]c(N)nc32)[C@H](F)[C@@H]1O)C(=O)OC1CCCCC1] | 4′-Azido-2′-deoxy-2′- fluoroguanosine-5′-{N,N′- bis[(S)-1-(cyclohex- oxycarbonyl)ethyl] phosphorodiamidate |
| I-62 | 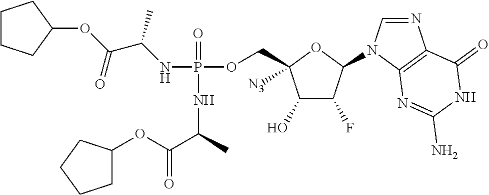 [SMILES: C[C@H](NP(=O)(N[C@@H](C)C(=O)OC1CCCC1)OC[C@@]1(N=[N+]=[N-])O[C@@H](n2cnc3c(=O)[nH]c(N)nc32)[C@H](F)[C@@H]1O)C(=O)OC1CCCC1] | 4′-Azido-2′-deoxy-2′- fluoroguanosine-5′-{N,N′- bis[(S)-1-(cyclopent- oxycarbonyl)ethyl] phosphorodiamidate |
| I-63 | 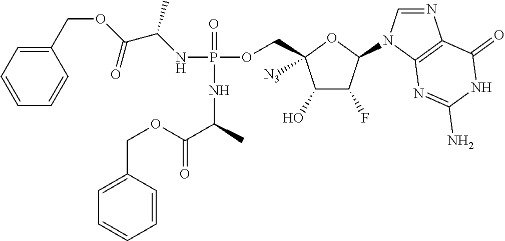 [SMILES: C[C@H](NP(=O)(N[C@@H](C)C(=O)OCc1ccccc1)OC[C@@]1(N=[N+]=[N-])O[C@@H](n2cnc3c(=O)[nH]c(N)nc32)[C@H](F)[C@@H]1O)C(=O)OCc1ccccc1] | 4′-Azido-2′-deoxy-2′- fluoroguanosine-5′-{N,N- bis[(S)-1- (benzyloxycarbonyl)ethyl] phosphorodiamidate |
| I-64 | 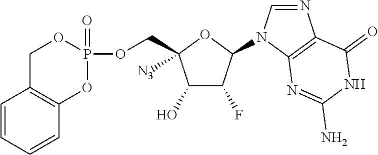 [SMILES: [N-]=[N+]=N[C@]1(COP2(=O)OCc3ccccc3O2)O[C@@H](n2cnc3c(=O)[nH]c(N)nc32)[C@H](F)[C@@H]1O] | 4′-Azido-2′-deoxy- 2′fluoro-5′-O-(2- oxido-4-H-1,3,2- benzodioxaphosphorin-2- yl)-guanosine |
| I-65 | 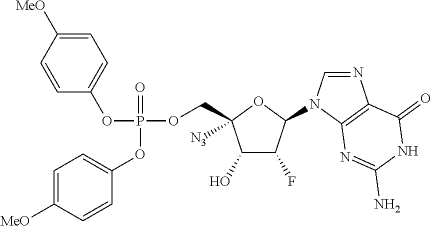 [SMILES: COc1ccc(OP(=O)(OC[C@@]2(N=[N+]=[N-])O[C@@H](n3cnc4c(=O)[nH]c(N)nc43)[C@H](F)[C@@H]2O)Oc2ccc(OC)cc2)cc1] | 4′-Azido-2′-deoxy- 2′-fluoro- 5′-O-[bis(4-methoxy- phenoxy)phosphinyl] guanosine |
| I-66 | 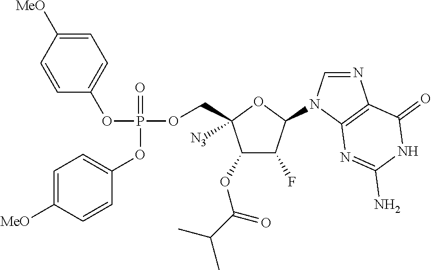 [SMILES: COc1ccc(OP(=O)(OC[C@@]2(N=[N+]=[N-])O[C@@H](n3cnc4c(=O)[nH]c(N)nc43)[C@H](F)[C@@H]2OC(=O)C(C)C)Oc2ccc(OC)cc2)cc1] | 3′-Acetyl-4′-azido- 2′-deoxy-2′-fluoro-5′- O-[bis(4-methoxy- phenoxy)phosphinyl] guanosine |
| I-67 | 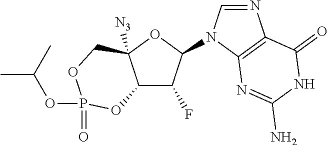 | 4′-Azido-2′-deoxy-2′- fluoroguanosine- 3′,5′-cyclic phosphoric acid isopropyl ester |

TABLE 2
Nucleoside triphosphates tested in Ebola polymerase assay.

| Compound Number | Structure |
|---|---|
| II-1 | 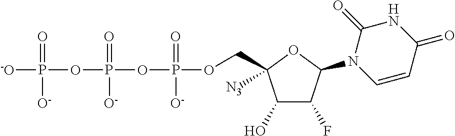 |
| II-2 | 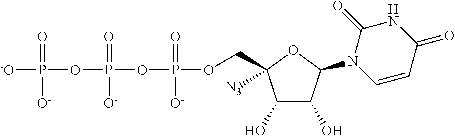 |
| II-3 | 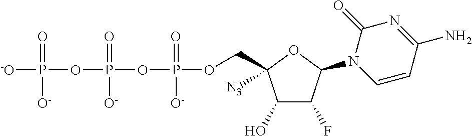 |
| II-4 | 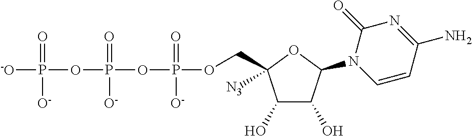 |
| II-5 | 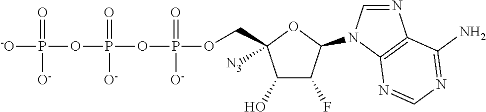 |
| II-6 | 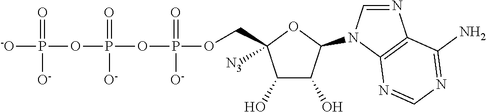 |
| II-7 | 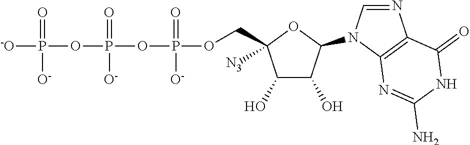 |
| II-8 | 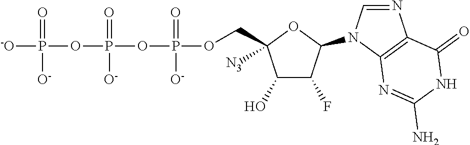 |

### EXAMPLES

Abbreviations used in this application include: acetyl (Ac), acetic acid (HOAc), azo-bis-isobutyrylnitrile (AIBN), 1-N-hydroxybenzotriazole (HOBt), atmospheres (Atm), high pressure liquid chromatography (HPLC), 9-borabicyclo[3.3.1]nonane (9-BBN or BBN), methyl (Me), tert-butoxycarbonyl (Boc), acetonitrile (MeCN), di-tert-butyl pyrocarbonate or boc anhydride (BOC₂O), 1-(3-dimethylaminopropyl)-3-ethylcarbodiimide hydrochloride (EDCI), benzoyl (Bz), benzyl (Bn), m-chloroperbenzoic acid (MCPBA), butyl (Bu), methanol (MeOH), benzyloxycarbonyl (cbz or Z), melting point (mp), carbonyl diimidazole (CDI), MeSO₂— (mesyl or Ms), 1,4-diazabicyclo[2.2.2]octane (DABCO), mass spectrum (ms) diethylaminosulfur trifluoride (DAST), methyl t-butyl ether (MTBE), dibenzylideneacetone (Dba), N-carboxyanhydride (NCA), 1,5-diazabicyclo[4.3.0]non-5-ene (DBN), N-bromosuccinimide (NBS), 1,8-diazabicyclo[5.4.0]undec-7-ene (DBU), N-methylmorpholine (NMM), N-methylpyrrolidone (NMP), 1,2-dichloroethane (DCE), pyridinium chlorochromate (PCC), N,N′-dicyclohexylcarbodiimide (DCC), pyridinium dichromate (PDC), dichloromethane (DCM), propyl (Pr), diethyl azodicarboxylate (DEAD), phenyl (Ph), di-iso-propylazodicarboxylate, DIAD, pounds per square inch (psi), di-iso-propylethylamine (DIPEA), pyridine (pyr), di-iso-butylaluminumhydride, DIBAL-H, room temperature, rt or RT, N,N-dimethyl acetamide (DMA), tert-butyldimethylsilyl or t-BuMe₂Si, (TBDMS), 4-N,N-dimethylaminopyridine (DMAP), triethylamine (Et₃N or TEA), N,N-dimethylformamide (DMF), triflate or CF₃SO₂-(Tf), dimethyl sulfoxide (DMSO), trifluoroacetic acid (TFA), 1,1′-bis-(diphenylphosphino)ethane (dppe), 2,2,6,6-tetramethylheptane-2,6-dione (TMHD), 1,1′-bis-(diphenylphosphino)ferrocene (dppf), thin layer chromatography (TLC), ethyl acetate (EtOAc), tetrahydrofuran (THF), diethyl ether (Et₂O), trimethylsilyl or Me₃Si (TMS), ethyl (Et), p-toluenesulfonic acid monohydrate (TsOH or pTsOH), lithium hexamethyl disilazane (LiHMDS), 4-Me-C₆H₄SO₂— or tosyl (Ts), iso-propyl (i-Pr), N-urethane-N-carboxyanhydride (UNCA), ethanol (EtOH). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford).

### General Conditions

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

4′-Azidouridine 4 and 4′-azidocytidine 5 were prepared in accordance to the literature (WO2002100414, WO2005000864 and J. Med. Chem., 2009, 52, 2971) and as shown in Scheme

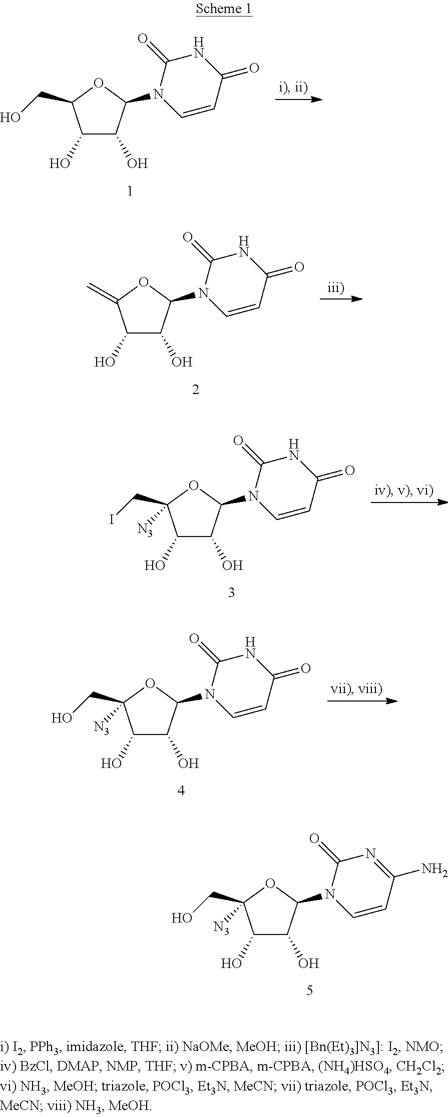 [SMILES: O=c1ccn([C@@H]2O[C@H](CO)[C@@H](O)[C@H]2O)c(=O)[nH]1 ; C=C1O[C@@H](n2ccc(=O)[nH]c2=O)[C@H](O)[C@@H]1O ; [N-]=[N+]=N[C@]1(CI)O[C@@H](n2ccc(=O)[nH]c2=O)[C@H](O)[C@@H]1O ; [N-]=[N+]=N[C@]1(CO)O[C@@H](n2ccc(=O)[nH]c2=O)[C@H](O)[C@@H]1O ; [N-]=[N+]=N[C@]1(CO)O[C@@H](n2ccc(N)nc2=O)[C@H](O)[C@@H]1O]

4′-Azido-2′-deoxy-2′-fluorocytidine 10 was prepared according to the literature (J. Med. Chem., 2009, 52, 2971) (Scheme 2).

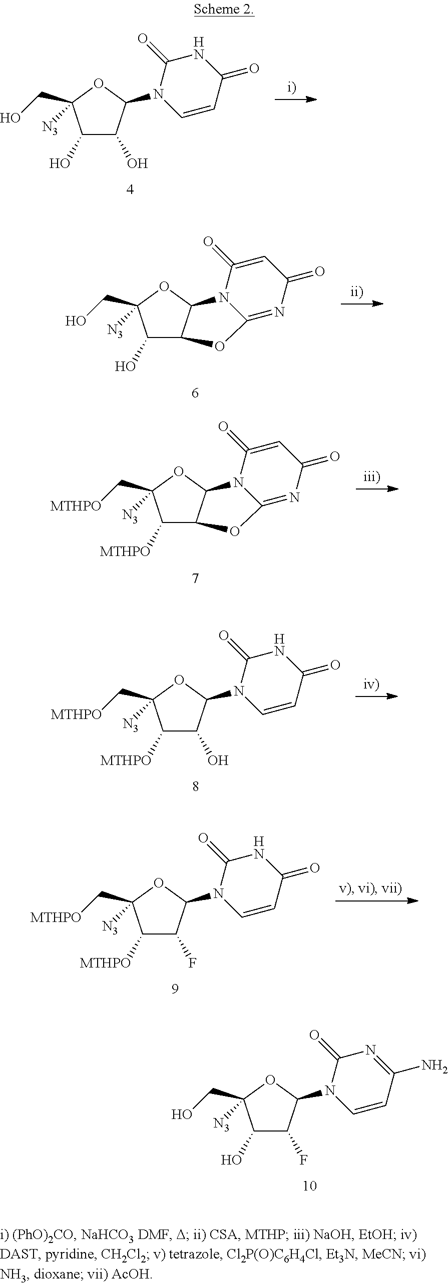

4′-azido-2′-deoxy-2′-fluorouridine 11 can be synthesized by treatment of the uridine intermediate 9 with acetic acid (Scheme 3).

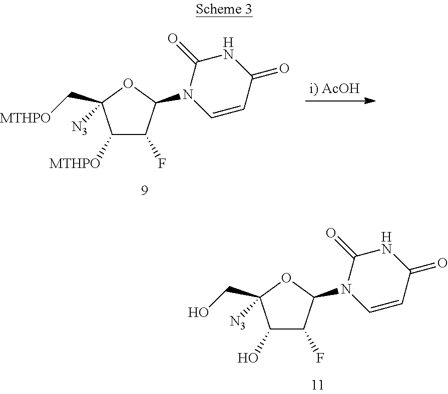

4′-Azido-2′-deoxy-2′-fluoroguanosine 16 is prepared in 10 steps from commercial 2′-deoxy-2′-fluoroguanosine 12 in accordance with the literature procedure (US2012070415) as outlined in Scheme 4.

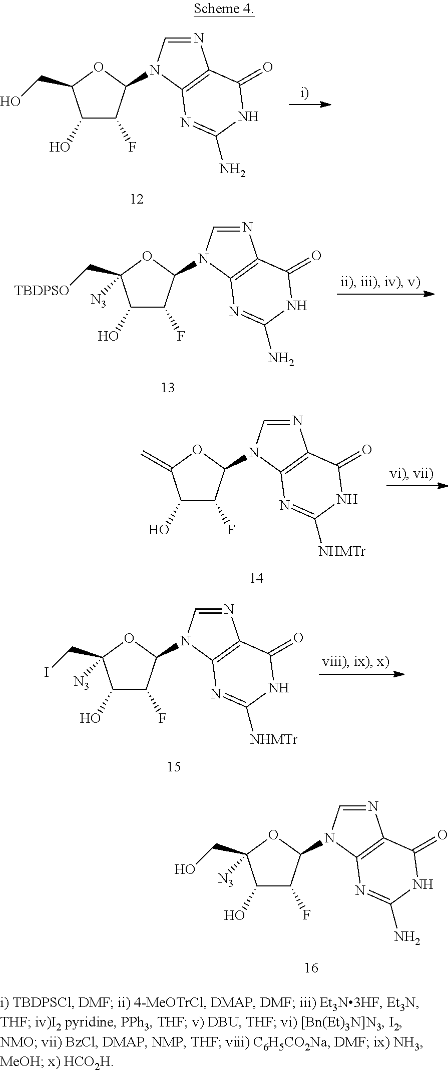

4′-Azido-2′-deoxy-2′-fluoroadenosine is prepared according to the literature (US2012070415) in 6 steps from commercial 2′-deoxy-2′-fluoroadenosine 17 as outlined in Scheme 5.

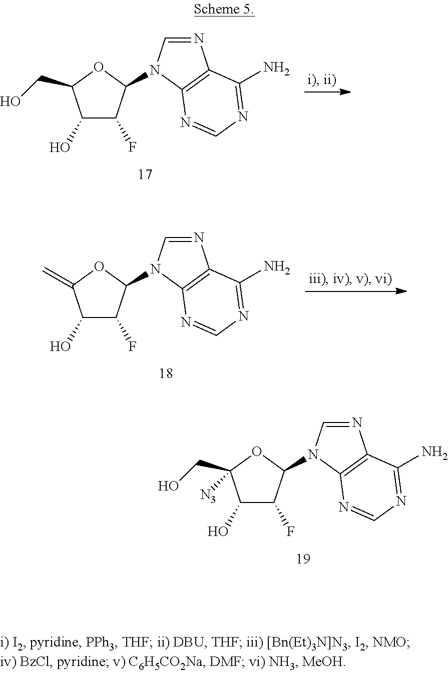 [SMILES: Nc1ncnc2c1ncn2[C@@H]1O[C@H](CO)[C@@H](O)[C@H]1F ; C=C1O[C@@H](n2cnc3c(N)ncnc32)[C@H](F)[C@@H]1O ; [N-]=[N+]=N[C@]1(CO)O[C@@H](n2cnc3c(N)ncnc32)[C@H](F)[C@@H]1O]

Phosphorochloridates, of type 22, were prepared according to the general procedure outlined in Scheme 6. Reaction of phosphorochloridates 22 with nucleosides provides phosphoramidates of type 24 (Scheme 7).

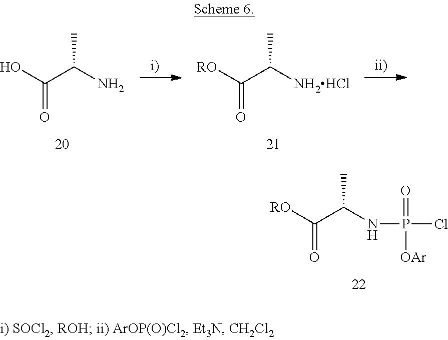

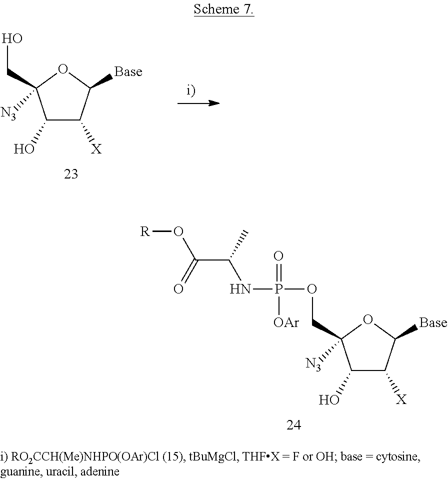

4′-Azidoadenosine (1-39) was prepared according to a literature procedure (J. Med. Chem., 2007, 50, 5463; WO2007020193) as outlined in Scheme 8.

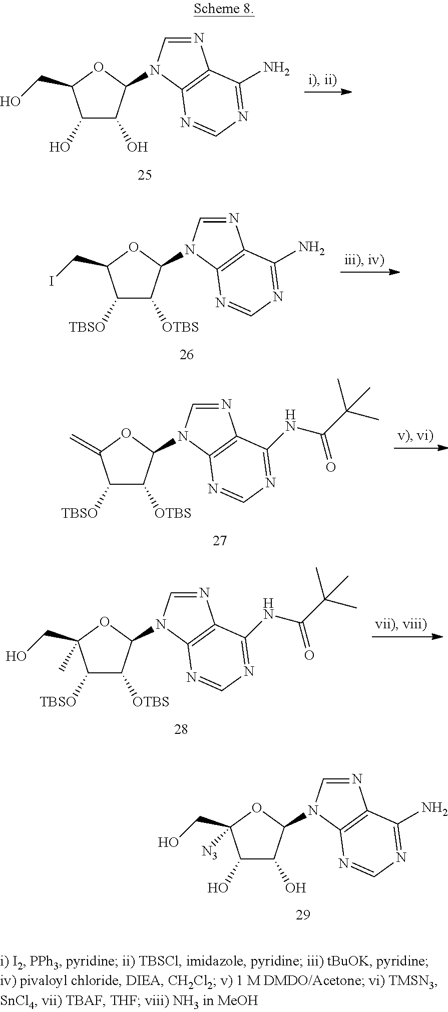 [SMILES: Nc1ncnc2c1ncn2[C@@H]1O[C@H](CO)[C@@H](O)[C@H]1O ; CC(C)(C)[Si](C)(C)O[C@@H]1[C@H](O[Si](C)(C)C(C)(C)C)[C@@H](CI)O[C@H]1n1cnc2c(N)ncnc21 ; C=C1O[C@@H](n2cnc3c(NC(=O)C(C)(C)C)ncnc32)[C@H](O[Si](C)(C)C(C)(C)C)[C@@H]1O[Si](C)(C)C(C)(C)C ; CC(C)(C)C(=O)Nc1ncnc2c1ncn2[C@@H]1O[C@](C)(CO)[C@@H](O[Si](C)(C)C(C)(C)C)[C@H]1O[Si](C)(C)C(C)(C)C ; [N-]=[N+]=N[C@]1(CO)O[C@@H](n2cnc3c(N)ncnc32)[C@H](O)[C@@H]1O]

4′-Azidoguanosine (1-48) 34 was prepared according to the literature procedure (US20120070415) as outlined in Scheme 9.

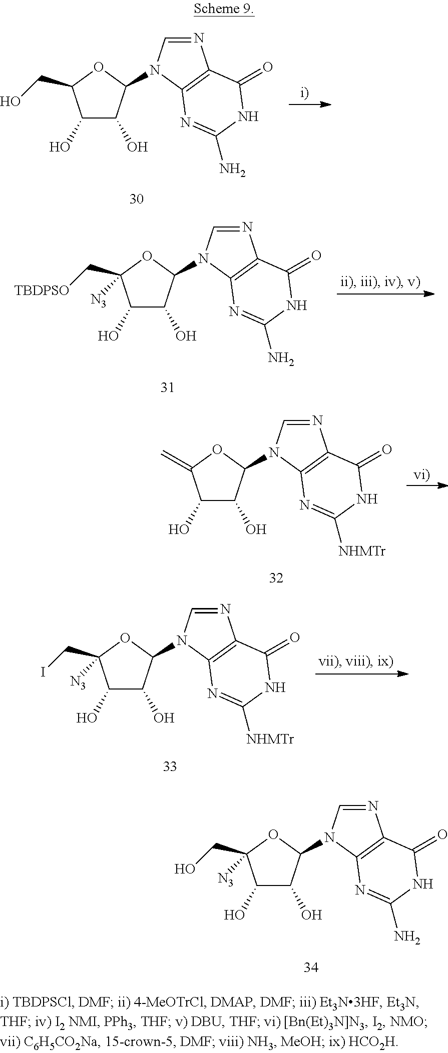

### General Procedure for Amino Ester Salts of Type 21.

Thionyl chloride (2 mol equiv.) was added drop-wise to a solution amino acid (20) (1 mol equiv) in alcohol (12 mol. equiv). After stirring at 0° C. for 2 h, the reaction mixture was warmed to room temperature and stirred for a further 24 h and then evaporated to dryness under reduced pressure. Trituration of the crude reside with diethyl ether provides the desired amino acid ester (21) as a hydrochloride salt.

### General Procedure for Phosphorochloridates of Type 22.

Phenyl phosphorodichloridate (1 mol equiv.) was added to a solution of the amino acid ester 21 (1 mol equiv.) in anhydrous dichloromethane at −78° C. Triethylamine (2 mol equiv.) was then added drop-wise. Once complete, the reaction mixture was allowed to warm to room temperature and then evaporated to dryness under reduced pressure. Purification by silica gel chromatography [petroleum ether:ethyl acetate (3:1)] provided Phosphorochloridates of type 22.

### General Procedure of Phosphoramidate Prodrugs of Type 24.

t-BuMgBr (1.6 mL, 1.6 mmol) was added to a cooled (0° C.) suspension of nucleosides of type 23 (1 mol equiv.) in dry THF (36 mL). The reaction mixture was left to stir under a nitrogen atmosphere for 30 min. The Phosphorochloridate of type 22 (2.5 mol equiv) was added drop-wise at 0° C. over 10 min. Once complete, the reaction mixture was stirred for 16 h at room temperature and then quenched with 1 mL MeOH. The resulting mixture was evaporated to dryness under reduced pressure. The phosphoramidates of type 24 were purified by column chromatography (10% methanol in dichloromethane).

### Biological Examples

The compounds of the invention and their isomeric forms and pharmaceutically acceptable salts thereof are useful in treating and preventing infections caused by Ebolavirus, Filovirus or related negative strand or positive strand RNA viruses, when used alone or when used in combination with other compounds targeting viral or cellular elements or functions involved in the virus lifecycle or when used in combination with immunomodulators or vaccines. Classes of compounds useful in the invention include, without limitation, all classes of antivirals.

| Compound | EC50 (μM)¹ | CC50 (μM) |
|---|---|---|
| I-6 | A | >50 |
| I-17 | A | >50 |
| I-47 | A | >50 |
| I-48 | B | >50 |

¹A (EC50 <20 μM); B (EC50 <50 μM)

Example of the measurement of antiviral activity. Antiviral activity can be measured as described by Uebelhoer L S et al. Antiviral Research 2014, 106:86-94. Infectious gLuc Ebola virus was generated in BHK-21 cells and used to infect Vero-E6 cells at an multiplicity of infection of 0.1. The level of viral replication was determined by the measurement of virus replication dependent secretion of Gaussia Luciferase into the cell culture supernatant. The level of virus replication was measured in the presence of different concentrations of test compounds and the data used to generate a dose response curve by non-linear fitting to a hyperbolic equation and to determine antiviral EC50 values.

Example of the measurement of antiviral activity. Antiviral activity can also be measured by the TCID50 method as described by Uebelhoer L S et al. Antiviral Research 2014, 106:86-94 The virus titers generated from virus infected Vero-E6 cells in the presence of different concentrations of test compound can be determined by the addition of supernatant samples of such cells to new Vero-E6 cells. The cells are then incubated for 5 day, before being fixed with formalin, permeabilized with Triton 0.1% and stained with a polyclonal antibody against the virus, followed by counter-staining with anti-rabbit Alexafluor 498 or 594 nm secondary antibodies (Molecular Probes). The TCID50/ml was determined using the Reed and Muench method.

Example of determination of cytotoxicity. The cytotoxicity of test compounds can be determined by determining the effect of different concentrations of test compound on the intracellular ATP levels in the infected and compound treated cells. The CellTiterGlo reagent (Promega) can be used to determine intracellular ATP levels.

It will be understood that references herein to treatment extend to prophylaxis as well as to the treatment of existing conditions, and that the treatment of animals includes the treatment of humans as well as other mammals. Furthermore, treatment of Ebolavirus, as used herein, also includes treatment or prophylaxis of a disease or a condition associated with or mediated by Ebolavirus infection, or the clinical symptoms thereof.

### Dosage and Administration:

The compounds of formula I have the potential to be efficacious as antiviral drugs for the treatment of Ebola infections in humans, or are metabolized to a compound that exhibit such activity.

In another embodiment of the invention, the active compound or its prodrug derivative or salt can be administered in combination with another antiviral agent, such as an anti-hepatitis agent, including those of formula I. When the active compound or its derivative or salt are administered in combination with another antiviral agent the activity may be increased over the parent compound. This can easily be assessed by preparing the derivative and testing its anti-Ebola activity according to the method described herein.

Administration of the active compound may range from continuous (intravenous drip) to several oral administrations per day (for example, Q.I.D) and may include oral, topical parenteral, intramuscular, intravenous, subcutaneous, transdermal (which may include a penetration enhancement agent), buccal and suppository administration, among other routes of administration.

The 4′-azido substituted nucleoside derivatives as well as their pharmaceutically useable salts, can be used as medicaments in the form of any pharmaceutical formulation. The pharmaceutical formulation can be administered enterally, either orally, e.g. in the form of tablets, coated tablets, dragées, hard and soft gelatine capsules, solutions, emulsions, syrups, or suspensions, or rectally, e.g. in the form of suppositories. They can also be administered parenterally (intramuscularly, intravenously, subcutaneously or intrasternal injection or infusion techniques), e.g. in the form of injection solutions, nasally, e.g. in the form of nasal sprays, or inhalation spray, topically and so forth.

For the manufacture of pharmaceutical preparations, the 4′-substituted nucleoside derivatives, as well as their pharmaceutically useable salts, can be formulated with a therapeutically inert, inorganic or organic excipient for the production of tablets, coated tablets, dragées, hard and soft gelatine capsules, solutions, emulsions or suspensions.

The compounds of formula I can be formulated in admixture with a pharmaceutically acceptable carrier. For example, the compounds of the present invention can be administered orally as pharmacologically acceptable salts. Because the compounds of the present invention are mostly water soluble, they can be administered intravenously in physiological saline solution (e.g., buffered to a pH of about 7.2 to 7.5). Conventional buffers such as phosphates, bicarbonates or citrates can be used for this purpose. Of course, one of ordinary skill in the art may modify the formulations within the teachings of the specification to provide numerous formulations for a particular route of administration without rendering the compositions of the present invention unstable or compromising their therapeutic activity. In particular, the modification of the present compounds to render them more soluble in water or other vehicle, for example, may be easily accomplished by minor modifications (salt formulation, esterification, etc.) which are well within the ordinary skill in the art. It is also well within the ordinary skill of the art to modify the route of administration and dosage regimen of a particular compound in order to manage the pharmacokinetics of the present compounds for maximum beneficial effect in patients.

For parenteral formulations, the carrier will usually comprise sterile water or aqueous sodium chloride solution, though other ingredients including those which aid dispersion may be included. Of course, where sterile water is to be used and maintained as sterile, the compositions and carriers must also be sterilized. Injectable suspensions may also be prepared, in which case appropriate liquid carriers, suspending agents and the like may be employed.

Suitable excipients for tablets, coated tablets, dragées, and hard gelatin capsules are, for example, lactose, corn starch and derivatives thereof, talc, and stearic acid or its salts.

If desired, the tablets or capsules may be enteric-coated or sustained release by standard techniques.

Suitable excipients for soft gelatine capsules are, for example, vegetable oils, waxes, fats, semi-solid and liquid polyols.

Suitable excipients for injection solutions are, for example, water, saline, alcohols, polyols, glycerin or vegetable oils.

Suitable excipients for suppositories are, for example, natural and hardened oils, waxes, fats, semi-liquid or liquid polyols.

Suitable excipients for solutions and syrups for enteral use are, for example, water, polyols, saccharose, invert sugar and glucose.

The pharmaceutical preparations of the present invention may also be provided as sustained release formulations or other appropriate formulations.

The pharmaceutical preparations can also contain preservatives, solubilizers, stabilizers, wetting agents, emulsifiers, sweeteners, colorants, flavourants, salts for adjustment of the osmotic pressure, buffers, masking agents or antioxidants.

The pharmaceutical preparations may also contain other therapeutically active agents known in the art.

The dosage can vary within wide limits and will, of course, be adjusted to the individual requirements in each particular case. For oral administration, a daily dosage of between about 0.01 and about 100 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 100 mg/kg body weight per day. A typical preparation will contain from about 5% to about 95% active compound (w/w). The daily dosage can be administered as a single dosage or in divided dosages, typically between 1 and 5 dosages per day.

In certain pharmaceutical dosage forms, the pro-drug form of the compounds, especially including acylated (acetylated or other) derivatives, pyridine esters and various salt forms of the present compounds are preferred. One of ordinary skill in the art will recognize how to readily modify the present compounds to pro-drug forms to facilitate delivery of active compounds to a target site within the host organism or patient. One of ordinary skill in the art will also take advantage of favorable pharmacokinetic parameters of the pro-drug forms, where applicable, in delivering the present compounds to targeted site within the host organism or patient to maximize the intended effect of the compound.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A method of treating a disease mediated by Ebolavirus, Marburgvirus, or other Filovirus, comprising administering to a patient in need thereof a therapeutically effective amount of a compound of Formula I 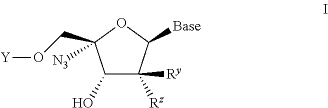
wherein:
Y is H or P(═X)(R′)(R);
R is O—R¹ or NHR^{1′};
R^{1′} is —C(R^{2a})(R^{2b})C(═O)OR³;
R′ is N(R⁴)C(R^{2a})(R^{2b})C(═O)OR³, —OP(═O)(OH)OH, —OP(═O)(OH)OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂;
each R⁴ is independently H, lower alkyl;
or R^{2b} and R⁴ together form (CH₂)₃;
R^{y}, and R^{z} are each independently H, Me, OH or F;
X is O or S; and
Base is uracil, cytosine, guanine, adenine, thymine, or heterocycloalkyl, each of which may optionally be substituted with one or more hydroxy, lower alkyl, lower alkoxy, halo, nitro or cyano;
or a pharmacologically acceptable salt thereof.

2. The method of claim 1, wherein the Filovirus is Ebolavirus.

3. The method of claim 1, wherein R^{z} is F.

4. A compound of Formula II, 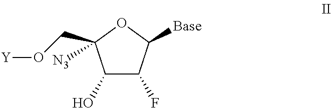
wherein:
Base is guanine;
Y is P(═X)(R′)(R);
R is O—R¹ or NHR^{1′};
R^{1′} is —C(R^{2a})(R^{2b})C(═O)OR³;
R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R⁴ together form CH₂; and
each R⁴ is independently H, lower alkyl;
or R^{2b} and R⁴ together form (CH₂)₃;
with the proviso that the compound is not cyclohexyl ((((2R,3R,4R,5R)-5-(2-amino-6-oxo-3,6-dihydro-9H-purin-9-yl)-2-azido-4-fluoro-3-hydroxytetrahydrofuran-2-yl)methoxy)(phenoxy)phosphoryl)-L-alaninate;
or a pharmacologically acceptable salt thereof.

5. The method of claim any one of claim 1, wherein the compound of Formula I is selected from the group consisting of:
4′-Azido-2′-deoxy-2′-fluorouridine;
4′-Azidouridine
4′-Azido-2′-deoxy-2′-fluorouridine-5′-(O-phenyl-N-(S)-1-(isopropoxycarbonyl)ethyl phosphoramidate
4′-Azidouridine-5′-(O-phenyl-N-(S)-1-(isopropoxycarbonyl)ethyl phosphoramidate;
4′-Azido-2′-deoxy-2′-fluorouridine-5′-(O-1-naphthyl-N-(S)-1-(isopropoxycarbonyl)ethyl phosphoramidate;
4′-Azidouridine-5′-(O-1-naphthyl-N-(S)-1-(benzyloxycarbonyl)ethyl phosphoramidate
4′-Azido-2′-deoxy-2′-fluorouridine-5′-(O-1-naphthyl-N-(S)-1-(i2,2-dimethylpropoxycarbonyl)ethyl phosphoramidate;
4′-Azido-2′-deoxy-2′-fluorouridine-5′-(O-1-naphthyl-N-(S)-1-(benzyloxycarbonyl)ethyl phosphoramidate;
4′-Azido-2′-deoxy-2′-fluorouridine-5′-(O-1-phenyl-N-(S)-1-(hexoxycarbonyl)ethyl phosphoramidate;
4′-Azido-2′-deoxy-2′-fluorouridine-5′-(O-1-naphthyl-N-(S)-1-(cyclopentoxycarbonyl)ethyl phosphoramidate;
4′-Azido-2′-deoxy-2′-fluorouridine-5′-{N,N′-bis[(S)-1-(isopropoxycarbonyl)ethyl]phosphorodiamidate};
4′-Azido-2′-deoxy-2′-fluorouridine-5′-{N,N′-bis[(S)-1-(benzyloxycarbonyl)ethyl]phosphorodiamidate};
4′-Azido-2′-deoxy-2′-fluorouridine-5′-{N,N′-bis[(S)-1-(hexoxycarbonyl)ethyl]phosphorodiamidate};
4′-Azido-2′-deoxy-2′-fluorouridine-5′-{N,N′-bis[(S)-1-(cyclopentoxycarbonyl)ethyl]phosphorodiamidate};
4′-Azido-2′-deoxy-2′-fluorouridine-5′-O-(2-oxido-4-H-1,3,2-benzodioxaphosphorin-2-yl)-uridine;
4′-Azido-2′-deoxy-2′-fluorouridine-5′-O-[bis(4-methoxyphenoxy)phosphinyl]uridine;
4′-Azido-2′-deoxy-2′-fluorocytidine;
4′-Azidocytidine;
4′-Azido-2′-deoxy-2′-fluorocytidine-5′-(O-phenyl-N-(S)-1-(ethoxycarbonyl)ethyl phosphoramidate;
4′-Azidocytidine-5′-(O-phenyl-N-(S)-1-(benzyloxycarbonyl)ethyl phosphoramidate;
4′-Azido-2′-deoxy-2′-fluorocytidine-5′-(O-phenyl-N-(S)-1-(isopropoxycarbonyl)ethyl phosphoramidate;
4′-Azidocytidine-5′-(O-phenyl-N-(S)-1-(eisopropoxycarbonyl)ethyl phosphoramidate;
4′-Azido-2′-deoxy-2′-fluorocytidine-5′-(O-phenyl-N-(S)-1-(neopentoxycarbonyl)ethyl phosphoramidate;
4′-Azido-2′-deoxy-2′-fluorocytidine-5′-(O-1-naphthyl-N-(S)-1-(isopropoxycarbonyl)ethyl phosphoramidate;
4′-Azido-2′-deoxy-2′-fluorocytidine-5′-(O-1-naphthyl-N-(S)-1-(2,2-dimethylpropoxycarbonyl)ethyl phosphoramidate;
4′-Azido-2′-deoxy-2′-fluorocytidine-5′-(O-1-naphthyl-N-(S)-1-(benzyloxycarbonyl)ethyl phosphoramidate;
4′-Azidocytidine-5′-(O-naphthyl-N-(S)-1-(benzyloxycarbonyl)ethyl phosphoramidate;
4′-Azido-2′-deoxy-2′-fluorocytidine-5′-(O-1-naphthyl-N-(S)-1-(3,3-dimethybutoxycarbonyl)ethyl phosphoramidate;
4′-Azido-2′-deoxy-2′-fluorocytidine-5′-(O-1-naphthyl-N-(S)-1-(cyclohexoxycarbonyl)ethyl phosphoramidate;
4′-Azido-2′-deoxy-2′-fluorocytidine-5′-(O-2-naphthyl-N-(S)-1-(isopropoxycarbonyl)ethyl phosphoramidate;
4′-Azido-2′-deoxy-2′-fluorocytidine-5′-{N,N′-bis[(S)-1-(isopropoxycarbonyl)ethyl]phosphorodiamidate};
4′-Azido-2′-deoxy-2′-fluorocytidine-5′-{N,N′-bis[(S)-1-(2,2-dimethylpropoxycarbonyl)ethyl]phosphorodiamidate};
4′-Azido-2′-deoxy-2′-fluorocytidine-5′-{N,N′-bis[(S)-1-(hexoxycarbonyl)ethyl]phosphorodiamidate};
4′-Azido-2′-deoxy-2′-fluorocytidine-5′-{N,N′-bis[(S)-1-(cyclohexoxycarbonyl)ethyl]phosphorodiamidate};
4′-Azido-2′-deoxy-2′-fluoro-5′-O-(2-oxido-4-H-1,3,2-benzodioxaphosphorin-2-yl)-cytidine;
4′-Azido-2′-deoxy-2′-fluoro-5′-O-[bis(4-methoxyphenoxy)phosphinyl]cytidine;
4′-Azido-2′-deoxy-2′-fluorocytidine-3′,5′-cyclic phosphoric acid isopropyl ester;
4′-Azido-2′-deoxy-2′-fluoroadenosine;
4′-Azidoadenosine;
4′-Azido-2′-deoxy-2′-fluoroadenosine-5′-(O-1-naphthyl-N-(S)-1-(isopropoxycarbonyl)ethyl phosphoramidate;
4′-Azidoadenosine-5′-(O-1-naphthyl-N-(S)-1-(isopropoxycarbonyl)ethyl phosphoramidate;
4′-Azido-2′-deoxy-2′-fluoroadenosine-5′-(O-2-naphthyl-N-(S)-1-(isopropoxycarbonyl)ethyl phosphoramidate;
4′-Azido-2′-deoxy-2′-fluoroadenosine-5′-{N,N′-bis[(S)-1-(isopropoxycarbonyl)ethyl]phosphorodiamidate;
4′-Azido-2′-deoxy-2′-fluoro-5′-O-(2-oxido-4-H-1,3,2-benzodioxaphosphorin-2-yl)-adenosine;
4′-Azido-2′-deoxy-2′-fluoro-5′-O-[bis(4-methoxyphenoxy)phosphinyl]adenosine;
4′-Azido-2′-deoxy-2′-fluoroadenosine-3′,5′-cyclic phosphoric acid isopropyl ester;
4′-Azido-2′-deoxy-2′-fluoroguanosine;
4′-Azidoguanosine;
4′-Azido-2′-deoxy-2′-fluoroguanosine-5′-(O-phenyl-N-(S)-1-(isopropoxycarbonyl)ethyl phosphoramidate;
4′-Azidoguanosine-5′-(O-phenyl-N-(S)-1-(isopropoxycarbonyl)ethyl phosphoramidate;
4′-Azido-2′-deoxy-2′-fluoroguanosine-5′-(O-1-naphthyl-N-(S)-1-(isopropoxycarbonyl)ethyl phosphoramidate;
4′-Azidoguanosine-5′-(O-1-naphthyl-N-(S)-1-(isopropoxycarbonyl)ethyl phosphoramidate;
4′-Azido-2′-deoxy-2′-fluoroguanosine-5′-(O-1-naphthyl-N-(S)-1-(hexoxycarbonyl)ethyl phosphoramidate;
4′-Azido-2′-deoxy-2′-fluoroguanosine-5′-(O-1-naphthyl-N-(S)-1-(isobutoxycarbonyl)ethyl phosphoramidate;
4′-Azido-2′-deoxy-2′-fluoroguanosine-5′-(O-1-naphthyl-N-(S)-1-(benzyloxycarbonyl)ethyl phosphoramidate;
3′-Acetyl-4′-azido-2′-deoxy-2′-fluoroguanosine-5′-(O-1-naphthyl-N-(S)-1-(isobutoxycarbonyl)ethyl phosphoramidate;
3′-Acetyl-4′-azido-2′-deoxy-2′-fluoroguanosine-5′-(O-1-naphthyl-N-(S)-1-(isopropoxycarbonyl)ethyl phosphoramidate;
3′-Acetyl-4′-azido-2′-deoxy-2′-fluoroguanosine-5′-(O-2-naphthyl-N-(S)-1-(isopropoxycarbonyl)ethyl phosphoramidate;
4′-Azido-2′-deoxy-2′-fluoroguanosine-5′-{N,N′-bis[(S)-1-(isopropoxycarbonyl)ethyl]phosphorodiamidate;
4′-Azido-2′-deoxy-2′-fluoroguanosine-5′-{N,N′-bis[(S)-1-(isobutoxycarbonyl)ethyl]phosphorodiamidate;
4′-Azido-2′-deoxy-2′-fluoroguanosine-5′-{N,N′-bis[(S)-1-(cyclohexoxycarbonyl)ethyl]phosphorodiamidate;
4′-Azido-2′-deoxy-2′-fluoroguanosine-5′-{N,N′-bis[(S)-1-(cyclopentoxycarbonyl)ethyl]phosphorodiamidate;
4′-Azido-2′-deoxy-2′-fluoroguanosine-5′-{N,N′-bis[(S)-1-(benzyloxycarbonyl)ethyl]phosphorodiamidate;
4′-Azido-2′-deoxy-2′fluoro-5′-O-(2-oxido-4-H-1,3,2-benzodioxaphosphorin-2-yl)-guanosine;
4′-Azido-2′-deoxy-2′-fluoro-5′-O-[bis(4-methoxyphenoxy)phosphinyl]guanosine;
3′-Acetyl-4′-azido-2′-deoxy-2′-fluoro-5′-O-[bis(4-methoxyphenoxy)phosphinyl]guanosine; and
4′-Azido-2′-deoxy-2′-fluoroguanosine-3′,5′-cyclic phosphoric acid isopropyl ester.

6. A method for treating an infection with a Filovirus related negative strand RNA virus from the Bornaviridae, Rhabdoviridae, Paramyxoviridae, Arenaviridae, Bunyaviridae or Orthomyxoviridae families, comprising administering to a patient in need thereof a therapeutically effective amount of a compound of Formula I 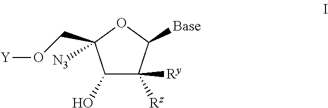
wherein:
Y is H or P(═X)(R′)(R);
R is O—R¹ or NHR^{1′};
R^{1′} is —C(R^{2a})(R^{2b})C(═O)OR³;
R′ is N(R⁴)C(R^{2a})(R^{2b})C(═O)OR³, —OP(═O)(OH)OH, —OP(═O)(OH)OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂;
each R⁴ is independently H, lower alkyl;
or R^{2b} and R⁴ together form (CH₂)₃;
R^{y}, and R^{z} are each independently H, Me, OH or F;
X is O or S; and
Base is uracil, cytosine, guanine, adenine, thymine, or heterocycloalkyl, each of which may optionally be substituted with one or more hydroxy, lower alkyl, lower alkoxy, halo, nitro or cyano;
or a pharmacologically acceptable salt thereof.

7. A method for treating an infection with a Filovirus related positive strand RNA virus from the Coronaviridae, Picornaviridae, Flaviviridae or Togaviridae families, comprising administering to a patient in need thereof a therapeutically effective amount of a compound of Formula I 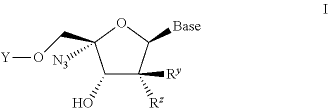
wherein:
Y is H or P(═X)(R′)(R);
R is O—R¹ or NHR^{1′};
R¹′ is —C(R^{2a})(R^{2b})C(═O)OR³;
R′ is N(R⁴)C(R^{2a})(R^{2b})C(═O)OR³, —OP(═O)(OH)OH, —OP(═O)(OH)OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂;
each R⁴ is independently H, lower alkyl;
or R^{2b} and R⁴ together form (CH₂)₃;
R^{y}, and R^{z} are each independently H, Me, OH or F;
X is O or S; and
Base is uracil, cytosine, guanine, adenine, thymine, or heterocycloalkyl, each of which may optionally be substituted with one or more hydroxy, lower alkyl, lower alkoxy, halo, nitro or cyano;
or a pharmacologically acceptable salt thereof.

8. A method for inhibiting replication of a Filovirus in a cell comprising administering to the cell a compound of Formula I 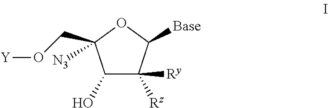
wherein:
Y is H or P(═X)(R′)(R);
R is O—R¹ or NHR^{1′};
R^{1′} is —C(R^{2a})(R^{2b})C(═O)OR³;
R′ is N(R⁴)C(R^{2a})(R^{2b})C(═O)OR³, —OP(═O)(OH)OH, —OP(═O)(OH)OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂;
each R⁴ is independently H, lower alkyl;
or R^{2b} and R⁴ together form (CH₂)₃;
R^{y}, and R^{z} are each independently H, Me, OH or F;
X is O or S; and
Base is uracil, cytosine, guanine, adenine, thymine, or heterocycloalkyl, each of which may optionally be substituted with one or more hydroxy, lower alkyl, lower alkoxy, halo, nitro or cyano;
or a pharmacologically acceptable salt thereof.

9. A compound of Formula II, 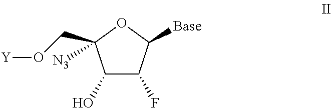
wherein:
Base is adenine;
Y is P(═X)(R′)(R);
R is O—R¹ or NHR^{1′};
R^{1′} is —C(R^{2a})(R^{2b})C(═O)OR³;
R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and
each R⁴ is independently H, lower alkyl;
or R^{2b} and R⁴ together form (CH₂)₃;
with the proviso that the compound is not isopropyl ((((2R,3R,4R,5R)-5-(6-amino-9H-purin-9-yl)-2-azido-4-fluoro-3-hydroxytetrahydrofuran-2-yl)methoxy)(phenoxy)phosphoryl)-L-alaninate;
or a pharmacologically acceptable salt thereof.

10. A compound of Formula II, 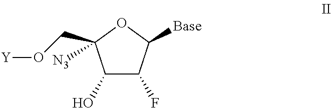
wherein:
Base is cytosine;
Y is P(═X)(R′)(R);
R is O—R¹ or NHR^{1′};
R^{1′} is —C(R^{2a})(R^{2b})C(═O)OR³;
R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and
each R⁴ is independently H, lower alkyl;
or R^{2b} and R⁴ together form (CH₂)₃;
with the proviso that the compound is not isopropyl ((((2S,3S,4S,5S)-5-(4-amino-2-oxopyrimidin-1(2H)-yl)-2-azido-4-fluoro-3-hydroxytetrahydrofuran-2-yl)methoxy)(((R)-1-isopropoxy-1-oxopropan-2-yl)amino)phosphoryl)-L-alaninate or isopropyl ((((2R,3R,4R,5R)-5-(4-amino-2-oxopyrimidin-1 (2H)-yl)-2-azido-4-fluoro-3-hydroxytetrahydrofuran-2-yl)methoxy)(phenoxy)phosphoryl)-L-alaninate;
or a pharmacologically acceptable salt thereof.

11. A compound of Formula II, 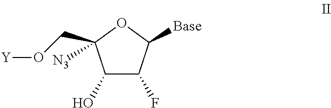
wherein:
Base is uracil;
Y is P(═X)(R′)(R);
R is O—R¹ or NHR^{1′};
R^{1′} is —C(R^{2a})(R^{2b})C(═O)OR³;
R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and
each R⁴ is independently H, lower alkyl;
or R^{2b} and R⁴ together form (CH₂)₃;
with the proviso that the compound is not isopropyl ((((2S,3S,4S,5S)-2-azido-5-(2,4-dioxo-3,4-dihydropyrimidin-1 (2H)-yl)-4-fluoro-3-hydroxytetrahydrofuran-2-yl)methoxy)(((R)-1-isopropoxy-1-oxopropan-2-yl)amino)phosphoryl)-L-alaninate, isopropyl ((((2R,3R,4S,5R)-2-azido-5-(2,4-dioxo-3,4-dihydropyrimidin-1(2H)-yl)-4-fluoro-3-hydroxytetrahydrofuran-2-yl)methoxy)(phenoxy)phosphoryl)-L-alaninate, or isopropyl ((((2R,3R,4R,5R)-2-azido-5-(2,4-dioxo-3,4-dihydropyrimidin-1(2H)-yl)-4-fluoro-3-hydroxytetrahydrofuran-2-yl)methoxy)(phenoxy)phosphoryl)-L-alaninate;
or a pharmacologically acceptable salt thereof.

12. A compound of Formula II, 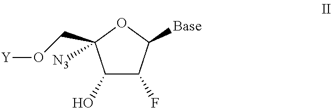
wherein:
Base is thymine;
Y is P(═X)(R′)(R);
R is O—R¹ or NHR^{1′};
R^{1′} is —C(R^{2a})(R^{2b})C(═O)OR³;
R′ is N(R⁴)C(R^{2a})(R^{2b})C(═O)OR³, —OP(═O)(OH)OH, or —OR³;
R¹ is H, lower haloalkyl, or aryl, wherein aryl is phenyl or naphthylenyl, optionally substituted with one or more lower alkyl, lower alkenyl, lower alkynyl, lower alkoxy, halo, lower haloalkyl, —N(R^{1a})₂, acylamino, —SO₂N(R^{1a})₂, —C(═O)R^{1b}, —SO₂(R^{1c}), —NHSO₂(R^{1c}), nitro, cyano, or R^{1″};
each R^{1a} is independently H or lower alkyl;
each R^{1b} is independently —OR^{1a} or —N(R^{1a})₂;
each R^{1c} is lower alkyl;
each R^{2a} and R^{2b} is independently H, lower alkyl, —(CH₂)ᵣN(R^{1a})₂, lower hydroxyalkyl, —CH₂SH, —(CH₂)S(O)ₚMe, —(CH₂)ₙNHC(═NH)NH₂, (1H-indol-3-yl)methyl, (1H-indol-4-yl)methyl, —(CH₂)ₘC(═O)R^{1b}, aryl and aryl lower alkyl, wherein aryl is optionally substituted with one or more hydroxy, lower alkyl, lower alkoxy, halo, nitro or cyano;
m is 0, 1, or 2;
n is 1, 2, or 3;
p is 1 or 2;
r is 1 or 2;
or R^{2a} is H and R^{2b} and R⁴ together form (CH₂)ₙ;
each R³ is independently H, lower alkyl, lower haloalkyl, phenyl or phenyl lower alkyl, wherein phenyl and phenyl lower alkyl are optionally substituted with lower alkoxy;
or R³ and R^{1″} together form CH₂; and
each R⁴ is independently H, lower alkyl;
or R^{2b} and R⁴ together form (CH₂)₃;
or a pharmacologically acceptable salt thereof.

13. A compound selected from the list consisting of:
4′-Azido-2′-deoxy-2′-fluorouridine-5′-(O-1-naphthyl-N-(S)-1-(isopropoxycarbonyl)ethyl phosphoramidate;
4′-Azido-2′-deoxy-2′-fluorouridine-5′-(O-1-naphthyl-N-(S)-1-(i2,2-dimethylpropoxycarbonyl)ethyl phosphoramidate;
4′-Azido-2′-deoxy-2′-fluorouridine-5′-(O-1-naphthyl-N-(S)-1-(benzyloxycarbonyl)ethyl phosphoramidate;
4′-Azido-2′-deoxy-2′-fluorouridine-5′-(O-1-phenyl-N-(S)-1-(hexoxycarbonyl)ethyl phosphoramidate;
4′-Azido-2′-deoxy-2′-fluorouridine-5′-(O-1-naphthyl-N-(S)-1-(cyclopentoxycarbonyl)ethyl phosphoramidate;
4′-Azido-2′-deoxy-2′-fluorouridine-5′-{N,N′-bis[(S)-1-(benzyloxycarbonyl)ethyl]phosphorodiamidate};
4′-Azido-2′-deoxy-2′-fluorouridine-5′-{N,N′-bis[(S)-1-(hexoxycarbonyl)ethyl]phosphorodiamidate};
4′-Azido-2′-deoxy-2′-fluorouridine-5′-{N,N′-bis[(S)-1-(cyclopentoxycarbonyl)ethyl]phosphorodiamidate};
4′-Azido-2′-deoxy-2′-fluorouridine-5′-O-(2-oxido-4-H-1,3,2-benzodioxaphosphorin-2-yl)-uridine;
4′-Azido-2′-deoxy-2′-fluorouridine-5′-O-[bis(4-methoxyphenoxy)phosphinyl]uridine;
4′-Azido-2′-deoxy-2′-fluorocytidine-5′-(O-phenyl-N-(S)-1-(ethoxycarbonyl)ethyl phosphoramidate;
4′-Azido-2′-deoxy-2′-fluorocytidine-5′-(O-phenyl-N-(S)-1-(neopentoxycarbonyl)ethyl phosphoramidate;
4′-Azido-2′-deoxy-2′-fluorocytidine-5′-(O-1-naphthyl-N-(S)-1-(isopropoxycarbonyl)ethyl phosphoramidate;
4′-Azido-2′-deoxy-2′-fluorocytidine-5′-(O-1-naphthyl-N-(S)-1-(2,2-dimethylpropoxycarbonyl)ethyl phosphoramidate;
4′-Azido-2′-deoxy-2′-fluorocytidine-5′-(O-1-naphthyl-N-(S)-1-(benzyloxycarbonyl)ethyl phosphoramidate;
4′-Azido-2′-deoxy-2′-fluorocytidine-5′-(O-1-naphthyl-N-(S)-1-(3,3-dimethybutoxycarbonyl)ethyl phosphoramidate;
4′-Azido-2′-deoxy-2′-fluorocytidine-5′-(O-1-naphthyl-N-(S)-1-(cyclohexoxycarbonyl)ethyl phosphoramidate;
4′-Azido-2′-deoxy-2′-fluorocytidine-5′-(O-2-naphthyl-N-(S)-1-(isopropoxycarbonyl)ethyl phosphoramidate;
4′-Azido-2′-deoxy-2′-fluorocytidine-5′-{N,N′-bis[(S)-1-(2,2-dimethylpropoxycarbonyl)ethyl]phosphorodiamidate};
4′-Azido-2′-deoxy-2′-fluorocytidine-5′-{N,N′-bis[(S)-1-(hexoxycarbonyl)ethyl]phosphorodiamidate};
4′-Azido-2′-deoxy-2′-fluorocytidine-5′-{N,N′-bis[(S)-1-(cyclohexoxycarbonyl)ethyl]phosphorodiamidate};
4′-Azido-2′-deoxy-2′-fluoro-5′-O-(2-oxido-4-H-1,3,2-benzodioxaphosphorin-2-yl)-cytidine;
4′-Azido-2′-deoxy-2′-fluoro-5′-O-[bis(4-methoxyphenoxy)phosphinyl]cytidine;
4′-Azido-2′-deoxy-2′-fluorocytidine-3′,5′-cyclic phosphoric acid isopropyl ester;
4′-Azido-2′-deoxy-2′-fluoroadenosine-5′-(O-1-naphthyl-N-(S)-1-(isopropoxycarbonyl)ethyl phosphoramidate;
4′-Azido-2′-deoxy-2′-fluoroadenosine-5′-(O-2-naphthyl-N-(S)-1-(isopropoxycarbonyl)ethyl phosphoramidate;
4′-Azido-2′-deoxy-2′-fluoroadenosine-5′-{N,N′-bis[(S)-1-(isopropoxycarbonyl)ethyl]phosphorodiamidate;
4′-Azido-2′-deoxy-2′-fluoro-5′-O-(2-oxido-4-H-1,3,2-benzodioxaphosphorin-2-yl)-adenosine;
4′-Azido-2′-deoxy-2′-fluoro-5′-O-[bis(4-methoxyphenoxy)phosphinyl]adenosine;
4′-Azido-2′-deoxy-2′-fluoroadenosine-3′,5′-cyclic phosphoric acid isopropyl ester;
4′-Azido-2′-deoxy-2′-fluoroguanosine-5′-(O-phenyl-N-(S)-1-(isopropoxycarbonyl)ethyl phosphoramidate;
4′-Azido-2′-deoxy-2′-fluoroguanosine-5′-(O-1-naphthyl-N-(S)-1-(isopropoxycarbonyl)ethyl phosphoramidate;
4′-Azido-2′-deoxy-2′-fluoroguanosine-5′-(O-1-naphthyl-N-(S)-1-(hexoxycarbonyl)ethyl phosphoramidate;
4′-Azido-2′-deoxy-2′-fluoroguanosine-5′-(O-1-naphthyl-N-(S)-1-(isobutoxycarbonyl)ethyl phosphoramidate;
4′-Azido-2′-deoxy-2′-fluoroguanosine-5′-(O-1-naphthyl-N-(S)-1-(benzyloxycarbonyl)ethyl phosphoramidate;
3′-Acetyl-4′-azido-2′-deoxy-2′-fluoroguanosine-5′-(O-1-naphthyl-N-(S)-1-(isobutoxycarbonyl)ethyl phosphoramidate;
3′-Acetyl-4′-azido-2′-deoxy-2′-fluoroguanosine-5′-(O-1-naphthyl-N-(S)-1-(isopropoxycarbonyl)ethyl phosphoramidate;
3′-Acetyl-4′-azido-2′-deoxy-2′-fluoroguanosine-5′-(O-2-naphthyl-N-(S)-1-(isopropoxycarbonyl)ethyl phosphoramidate;
4′-Azido-2′-deoxy-2′-fluoroguanosine-5′-{N,N′-bis[(S)-1-(isopropoxycarbonyl)ethyl]phosphorodiamidate;
4′-Azido-2′-deoxy-2′-fluoroguanosine-5′-{N,N′-bis[(S)-1-(isobutoxycarbonyl)ethyl]phosphorodiamidate;
4′-Azido-2′-deoxy-2′-fluoroguanosine-5′-{N,N′-bis[(S)-1-(cyclohexoxycarbonyl)ethyl]phosphorodiamidate;
4′-Azido-2′-deoxy-2′-fluoroguanosine-5′-{N,N′-bis[(S)-1-(cyclopentoxycarbonyl)ethyl]phosphorodiamidate;
4′-Azido-2′-deoxy-2′-fluoroguanosine-5′-{N,N′-bis[(S)-1-(benzyloxycarbonyl)ethyl]phosphorodiamidate;
4′-Azido-2′-deoxy-2′fluoro-5′-O-(2-oxido-4-H-1,3,2-benzodioxaphosphorin-2-yl)-guanosine;
4′-Azido-2′-deoxy-2′-fluoro-5′-O-[bis(4-methoxyphenoxy)phosphinyl]guanosine;
3′-Acetyl-4′-azido-2′-deoxy-2′-fluoro-5′-O-[bis(4-methoxyphenoxy)phosphinyl]guanosine; and
4′-Azido-2′-deoxy-2′-fluoroguanosine-3′,5′-cyclic phosphoric acid isopropyl ester.

14. A compound selected from the group consisting of:
4′-Azido-2′-deoxy-2′-fluorouridine-5′-(O-phenyl-N-(S)-1-(isopropoxycarbonyl)ethyl phosphoramidate;
4′-Azido-2′-deoxy-2′-fluorouridine-5′-{N,N′-bis[(S)-1-(isopropoxycarbonyl)ethyl]phosphorodiamidate};
4′-Azido-2′-deoxy-2′-fluorocytidine-5′-(O-phenyl-N-(S)-1-(isopropoxycarbonyl)ethyl phosphoramidate; and
4′-Azido-2′-deoxy-2′-fluorocytidine-5′-{N,N′-bis[(S)-1-(isopropoxycarbonyl)ethyl]phosphorodiamidate}.

15. A compound of Formula I, wherein the compound is 4′-Azidouridine-5′-(O-1-naphthyl-N-(S)-1-(benzyloxycarbonyl)ethyl phosphoramidate.

16. A compound of Formula I, wherein the compound is selected from the group consisting of:
4′-Azidouridine-5′-(O-phenyl-N-(S)-1-(isopropoxycarbonyl)ethyl phosphoramidate;
4′-Azidocytidine-5′-(O-phenyl-N-(S)-1-(benzyloxycarbonyl)ethyl phosphoramidate;
4′-Azidocytidine-5′-(O-phenyl-N-(S)-1-(eisopropoxycarbonyl)ethyl phosphoramidate;
4′-Azidocytidine-5′-(O-naphthyl-N-(S)-1-(benzyloxycarbonyl)ethyl phosphoramidate;
4′-Azidoadenosine-5′-(O-1-naphthyl-N-(S)-1-(isopropoxycarbonyl)ethyl phosphoramidate;
4′-Azidoguanosine-5′-(O-phenyl-N-(S)-1-(isopropoxycarbonyl)ethyl phosphoramidate; and
4′-Azidoguanosine-5′-(O-1-naphthyl-N-(S)-1-(isopropoxycarbonyl)ethyl phosphoramidate.
